(19) United States
(12) Patent Application Publication
(10) Pub. No.: US 2017/0340756 A1
(43) Pub. Date: Nov. 30, 2017

(54) PHARMACEUTICAL FORMULATION HAVING REVERSE THERMAL GELATION PROPERTIES FOR LOCAL DELIVERY OF NANOPARTICLES

(71) Applicant: Exchange Imaging Technologies GmbH, Darmstadt (DE)

(72) Inventor: Greb Wolfgang, Düsseldorf (DE)

(21) Appl. No.: 15/533,125

(22) PCT Filed: Dec. 6, 2015
(86) PCT No.: PCT/EP2015/078741

§ 371 Date: Jun. 5, 2017

(30) Foreign Application Priority Data
Dec. 5, 2014 (EP) 14196674.7

(51) Int. Cl. A61K 49/00; A61K 9/00; A61K 9/50; A61K 9/51

(52) U.S. Cl. CPC A61K 49/0067; A61K 9/5115; A61K 9/0019; A61K 9/501; A61K 9/5031

(57) ABSTRACT

The present invention refers to a pharmaceutical formulation for injection comprising fluorescent nanoparticles as in vivo diagnostics. The present invention relates to an injectable pharmaceutical formulation for human medicine and/or veterinary use, comprising 17% to 20% per weight of poloxamer 407 and 3%-15% per weight of poloxamer 188, 0.10 nM to 10.0 μM fluorescent nanoparticles and water or an aqueous buffer, wherein the pharmaceutical formulation is liquid at 4° C.-32° C. and forms a gel at about 37° C., their use as an in vivo marker and methods of their preparation. The inventive formulation is useful for local control and prevention of spreading/diffusion of nanoparticles, and thus allows full utilization of their quantum physics properties for example as a tool to enable surgical precision of tumor removal; even without tumor specific epitope binding antibodies.

The present invention refers to a pharmaceutical formulation, preferably for injection and for human medicine and/or veterinary use, comprising 17% to 20% per weight of poloxamer 407 and 3%-15% per weight of poloxamer 188, 0.001-0.15% per weight nanoparticles and water, wherein the formulation is liquid at 4° C.-32° C. and forms a gel at 37° C. Furthermore, the present invention refers to the in vivo medical use of the inventive formulation and methods of their preparation.

### BACKGROUND OF THE INVENTION

Nanoparticles (NPs) have various potential uses in medicine such as diagnostic imaging or labelling, drug targeting, and therapeutic applications like tumor destruction by electronic field heating using magnetic iron nanoparticles. Nanoparticles are favourably used because of their small size but their high diffusion in biological tissues, especially that of small nanoparticles sized 1-20 nm, can be unwanted and present a problem when local retention and concentration maintenance at the site of local application is needed. Typically, after bodily applications of small NPs they quickly and randomly diffuse in and out of tissues and vessels and distribute into various organs. This poses the general problem of controlling their location and effects in respect to their desired use, local dosing and effective concentration, undesired effects, lack of effectiveness, uncontrollable distribution and remote accumulation.

When nanoparticles are applied to the body in solution and/or classical pharmaceutical formulations they typically diffuse very quickly due to themselves being much smaller than biological structures and the surrounding anatomy: they easily diffuse through tissue and cellular barriers, exit from blood vessels, enter cells, show unwanted accumulation in certain organs or are eliminated by active processes like macrophage capture. This should be prevented by a special formulation.

Colorectal carcinoma (CRC) constitutes a major health problem. It has been estimated that worldwide 500,000 patients per year will die due to this malignancy. In Germany alone, CRC accounts for about 27,000 mortalities per year. While 90% of patients with localized cancer will survive the next 5 years, this number decreases to about 10% in patients with metastasized disease.

In order to reduce mortality and morbidity, early detection of colorectal cancer is important and to achieve this goal, various national medical guidelines recommend regular screening programs. Colonoscopy plays an important role, both, in cancer prevention and cancer diagnosis, and is the gold standard in CRC detection. While open surgery has long been the method of choice for resection of cancerous lesions, in recent years, minimal invasive operation techniques have become increasingly popular. Various studies performed could evidence that laparoscopy has no disadvantage in the long term compared to open surgery while having various short term benefits. Since the surgeon loses the ability to palpate tumours during a laparoscopic procedure, a proper localization of cancerous lesions prior to surgery is of very high importance. It has been shown that colonoscopy alone is not sufficient for this purpose as in up to 20% of cases the localization of the tumour based on current markers available turns out to be erroneous, ultimately resulting in conversion i.e. a switch from a planned laparoscopy to open surgery. Various guidelines strongly recommend endoscopic tumour location marking prior to surgical tissue removal. Established methods include the use of metal clips and tattooing with ink or dyes. While these tools like India Ink are commonly considered safe if used in a sterile and diluted form, they leave ample room for improvement of the products' features, also to surmount the so far unaddressed associated use-drawbacks as described in literature.

Cognizant of the currently available product drawbacks in tissue-marking, most of which primordially associated with poor, post marking, area recognition the present invention provides a simple to use, but highly effective tissue marker.

The present invention relates—for example—to a method to reduce diffusion and keep fluorescent NPs at the site of application, especially of manually targeted applications (such as endoscopic or deep intra-tumoural injections, in situ topical mucosal and skin applications, etc). Therefore a formulation showing in situ gel formation due to temperature change has been developed. Such formulation allows liquid application and injection at room temperature and, through local gel formation at body respective tissue temperature at 37° C., a local retention for a determined and required period of time. This then effectively allows use of nanoparticle in a desired concentrated manner utilizing the typical nanoparticle effects (such as quantum physics and quantum chemistry effects, like strong fluorescence and paramagnetism or multimodal combinations of both) of concentrated groups of NPs, which differentiates nanoparticles from larger particles. In light of the toxic potential of nanoparticles, pharmaceutical formulations should keep the nanoparticles locally, especially to allow for surgical removal together with the tumour tissue during the dissection.

The present invention allows usage of nanoparticles as tool for surgical precision of tumour removal even without tumor specific epitope binding antibodies. The inventive formulation is particularly designed for in vivo use in human medicine. The inventive formulation that promotes retention and higher local NP concentration is highly desirable for certain applications, prevents NP-aggregation (NP-aggregation negatively affects fluorescence), and stabilizes the wanted quantum effect and medical utility. Particularly body movements during diagnostic or therapeutic procedures like movements of muscle (via breathing or walking or change of body position for imaging) and or gastrointestinal peristalsis facilitate diffusion. Therefore a formulation containing nanoparticles, and especially small quantum dots, showing less diffusion but being easily injectable is particularly useful under these conditions requiring local application stability of the nanoparticles.

In the following a typical example for the usage of such a formulation is described:

When endoscopic removal of early found or pre-malignant colon cancer lesions is not possible, the removal of these lesions and in particular all malignant appearing lesions is the domain of minimally invasive surgery. However, the intraoperative location and identification of lesions previously detected by gut endoscopy is often difficult, particularly during laparoscopic surgery, where the surgeon has no other chance than visual inspection. Therefore, in recent years, numerous attempts have been made to establish methods to highlight areas to be resected by endoscopic or laparoscopic markers prior to surgery, mainly during pre-operative endoscopy. An ideal marker would be visible from both sides of the gut wall (transluminal marker).

The lack of accurate lesion localization and identification during laparoscopy may lead to start open surgery during the session (called conversion) with substantial time delay or resection of the wrong segment of bowel. Endoscopic tattooing is one of useful tool for the localization of small colorectal lesions especially in the laparoscopic setting. Thereby a sterile, non-pyrogenic ink (suspensions of carbon particles) is used as an endoscopic marker for marking polyps and lesions in the gastrointestinal tract. Other dyes that have been assessed as endoscopic markers include methylene blue, indigo carmine, hematoxylin, eosin, and indocyanine green (ICG). However, some studies have revealed complications resulting from this procedure, such as a very rapid diffusion into the surrounding tissue, dye bleaching, or a rapid fading. Furthermore in Europe, the use of ink is limited by the lack of approval for medical purposes. There is only one product being CE-certificated, namely Spot™ by GI Supply. Alternative localization techniques have included metal clips and barium enema, however fluorescent markers in the visible range would be of great advantage for endoscopic/laparoscopic optical technologies, especially in minimal invasive surgery.

Fluorescent Nanoparticles such as dye doped silica- or calcium phosphate-particles or surface modified semiconductor particles like those made of binary compounds such as lead sulfide, lead selenide, cadmium selenide, cadmium sulfide or cadmium telluride or from ternary compounds such as cadmium selenide sulfide are used in biological research as imaging agents; for clinical/human use as location markers or fiducials (X-ray detection of NPs in human) carbon nanoparticle (Indian Ink) and gold nanoparticles have been used, respectively. For instance, such Qdots® or quantum dots are used because their fluorescence in terms of intensity and photostability exceeds the one of most other known fluorescence dyes. A direct, predictable relationship exists between the physical size of the quantum dots and the energy of the exciton and therefore, the wavelength and color of the emitted light. The small diameter between 2 and 20 nm allows using of quantum dots for different diagnostic purposes in in vitro studies. Nanoparticles and especially quantum dots coupled to proteins, oligonucleotides, small molecules, etc., may be used for direct binding of the nanoparticles to cellular targets and receptors of interest.

Recently many new light emitting nanoparticles have been described, not only semiconductor particles but also dye doped particles. For example, WO 2010/030119 A2 and WO 2011/109214 A2 disclose fluorescent nanoparticles based on silica and Calcium phosphate. Nevertheless, the intensity and stability of their fluorescence is inferior compared to particles containing metal ions such as cadmium or zinc as semiconductor material. Cadmium free quantum dots/nanoparticles may contain zinc, calcium, magnesium, aluminum, gallium or indium or iron oxide particles made fluorescent by surface modification.

In general, the toxic potential, rapid diffusion and or organic distribution pattern, elimination/excretion kinetics of these nanoparticles as well as the amounts to be used has significantly limited their medical use in humans and especially their use for endoscopic marking of lesions. One of the main disadvantages of dyes used in vivo and especially of nanoparticles, due to their small size, is a rapid diffusion into the surrounding tissue or their uptake into and subsequently distribution by the blood system. Thus, in vivo there is only limited time period after staining e.g. for surgical removal once a tissue area has been marked. Diminished fluorescence due to diffusion requires injecting a higher dose, which is undesired in man because of toxic potential.

The inventive formulation containing special cell and tissue binding water soluble nanoparticles, and particularly fluorescent nanoparticles, and more particularly quantum dots, showing less diffusion but being easily applicable, particularly easily injectable is, therefore, advantageous. The formulation provides a prolonged staining of the local site and control of the injection and/or reduces the systemic exposure to possibly hazardous materials in living animal or man, such as cadmium containing quantum dots. Ideally, such foreign and toxic marker is to be removed and cut out by the surgeon with the tissue to be excised, at least when the marker does not consist of naturally occurring ingredients.

In another example for therapeutic local tissue targeting, ferrofluids (colloidal liquids made of super paramagnetic nanoparticles) are used for organ targeting and need to be held at the bodily target site to keep the concentration high at this—typically a tumor—position. In such “magnetic field” treatment the iron nanoparticles are heated by externally applied electric fields, with the consequence of local cell destruction. Magnetic nanoparticles are further suitable for MRI (magnetic resonance image) diagnostics. Application of such iron oxide nanoparticles is also improved by using an inventive formulation containing such super paramagnetic nanoparticles. Such particles can be of special value if also coated by fluorescent surface passivation to enable multimodal visible detection by the surgeon for and during operation like tumor resection.

Examples for suitable super paramagnetic nanoparticles are γ-Fe₂O₃, Fe₃O₄, MnFe₂O₄, NiFe₂O₄, CoFe₂O₄, and any mixtures thereof. The two main forms are magnetite (Fe₃O₄) and its oxidized form maghemite (γ-Fe₂O₃). For example, WO 01/74337 A1 and EP 1378239 A1 disclose super paramagnetic nanoparticles which may be part of a formulation according to the invention.

Furthermore, photothermal therapy (PTT) is extensively investigated as an alternative procedure to treat cancer. In PTT, heat is generated within the targeted carcinoma tissue through absorption of applied laser light, causing thermal injury and cell death. Thereby the light sensitivity of targeted tissue is increased by use of dye (particularly chromophores absorbing light in the near infrared (NIR) region of the electromagnetic spectrum) doped nanoparticles such as gold nanoparticles, gold nanorods, gold nanoshells (silica particles coated with a nanometer-thick gold shell), nanoparticles containing organic chromophores like indocyanine green (ICG), and silver/organic dye composites nanoparticles. The use of nanoparticles absorbing and emitting at eye visible wavelengths with tumor seeking ligands—as the quantum dots described above—will improve this technique, as the operator will see the tumor cells to adjust the laser equipment. For both tumor-imaging and photodynamic anticancer therapy applications, the delivery of chromophore (such as ICG) containing or direct fluorescent nanoparticles to the tumor site and accumulation and retention in this site are the crucial steps.

In prior art it is known to use formulations based on polymers being liquid at room temperature and, through local gel formation at body respective tissue temperature at 37° C., to form a local retention depot of the drug for a prolonged drug release. WO 2008/041246 A2 discloses injectable formulations of the active agents/drugs tamsulosin and letrozole used as micro- or nanoparticle. WO 03/008376 A2 discloses L-DOPA containing compounds, such as poloxamers linked to L-DOPA. It is further disclosed that these polymers have a sol-gel transition at specified temperatures. WO 03/008376 A2 teaches also that different parameters influences said specific temperature, such as length of poly(ethylenglycol)- and poly(propylenglycol)-blocks but also further ingredients and derivatization of the polymers. The present invention provides for the first time an optimized formulation of fluorescent dye, namely solid particle quantum dots, being liquid at 4° C.-30° C. and preferably 4° C.-32° C. and forming a gel at 37° C. Thus, the pharmaceutical formulation of the present invention containing the fluorescent quantum dots (Qdots) is liquid until 30° C. or is liquid at 30° C. and preferably at 32° C. and is a gel or is gel-like or gelatinous at 37° C. and above. Between 30° C. and 37° C. and preferably between 32° C. and 37° C. the formulation changes the aggregate state from liquid to gelatinous or gel-like. The terms “gelatinous” and “gel-like” are used synonymous herein. Moreover tests have shown that above 37° C. and up to 42° C. the formulation will remain gelatinous or gel-like while the gel will not become harder. In addition, the inventive formulation provides firstly, a formulation of a fluorescent dye suitable for use as a marker of gastrointestinal tumor areas applied by injection during an endoscopic or surgical procedure. Before, it was not apparent that a formulation containing fluorescent nanoparticles and being liquid at 4° C.-32° C. and forming a gel at 37° C. is particularly suitable for endoscopic tumour location marking prior to surgical tissue removal.

In contrast to the above intention of release of chemical drugs in form of nanoparticle it is the objective of the present invention to provide a pharmaceutical formulation which allows convenient/easy fluid injection of solid nanoparticles to exert their own physical effects but having less diffusion resulting in a prolonged and controlled property, i.e. fluorescence, of the nanoparticles at the site of local deposition on, at and/or in the tissue of a living body, preferably into cancer prone tissues and in particular in the gastrointestinal system, after local application, implantation and or injection.

The objective of the present invention is solved by the teaching of the independent claims. Further advantageous features, aspects and details of the invention are evident from the dependent claims, the description, the figures, and the examples of the present application.

### DESCRIPTION OF THE INVENTION

To avoid the rapid decay of fluorescence when marking tumour location during endoscopic procedure and prior to surgical tissue removal a novel pharmaceutical formulation was envisaged. It could be found that a composition of fluorescent nanoparticles having liquid state at low temperatures (for storage and injection) while it becomes a gel like status at body temperature is very suitable because it increases local retention time in the target tissue after application.

The inventors could surprisingly show that nanoparticles, such as quantum dots, embedded in a pharmaceutical formulation containing poloxamers and an aqueous excipient remain longer at the site of injection, while unformulated nanoparticles quickly diffuse into the surrounding tissue and are rapidly taken up by the blood and lymphatic system. The term “unformulated nanoparticles” refers to nanoparticles which are dissolved or suspended in water or an aqueous buffer without further ingredients and especially without glycol oligomers or glycol polymers. The term “glycol oligomers or glycol polymers” refers to oligomers or polymers containing glycol units —O—CH₂—CH₂—O—.

Therefore, the present invention relates to a pharmaceutical formulation for injection comprising 5% to 50%, preferred 10% to 40% per weight of at least one poloxamer, 0.10 nM to 10.0 μM of at least one nanoparticle, preferably 1.0 nM to 6.0 μM, more preferably 50.0 nM to 2.0 μM and particularly preferred 10.0 nM to 1.0 μM of at least one nanoparticle and water or an aqueous buffer, wherein the pharmaceutical formulation is liquid at 4° C.-32° C., preferably at 4° C.-25° C., more preferably at 4° C.-20° C. and forms a gel at above 32° C. to 37° C. (being semisolid and remaining at such viscosity above 42° C.). The nanoparticles are preferably ferromagnetic or fluorescent. The pharmaceutical formulations disclosed herein are intended for human medicine and/or veterinary use. In between the temperature range of about 25-32° C. to about 37° C. the pharmaceutical formulation is in the intermediate state of becoming more and more gel like with increasing temperature and less fluid.

It is preferred that the pharmaceutical formulation of the present invention is liquid at 4° C.-30° C., preferably 4° C.-32° C. and forms a gel at above 32° C. to below 37° C. body temperature. Thus, the pharmaceutical formulation of the present invention is liquid until 30° C. or is liquid at 30° C. and is a gel or is gel-like or gelatinous at 37° C. and above. Between 30° C. and 37° C. and preferably between 32° C. and 37° C. the formulation changes the aggregate state from liquid to gelatinous or gel-like. The terms “gelatinous” and “gel-like” are used synonymous herein. Thereby the pharmaceutical formulation of the present invention should be semisolid or gel-like within 30 minutes, preferably within 10 minutes, and even more preferred within 2 minutes, after administration, which means at body temperature and at a pH in the range of 7-8. Thereby being semisolid or gel-like means reaching a viscosity at D100 sec-1 of 1.0 Pa·s or up to 3.0 Pa s at 37° C. The viscosity at shear rate D100 sec-1 at 5° C. and up to 32° C. should be between 0.1 and 0.3 Pas, thus leaving a difference between the two obtained temperature viscosity values of at least a factor of 5 or more.

In addition the pharmaceutical formulation of the present invention should not become semisolid or gel-like by shear stress only. This is important for injectability of the formulation. Ideally, the transition from liquid to gel should only be caused by a change in temperature from room temperature to body temperature. Further viscosity increase, especially above fever temperatures, should be prevented as solidity of resident material should not hinder and impinge bodily tissue movements, like gut peristalsis or muscle contractions.

A preferred embodiment of the present invention refers to a pharmaceutical formulation, preferably for injection and for human medicine and/or veterinary use, comprising 17% to 20% per weight of poloxamer 407 and 3%-15% per weight of poloxamer 188, 0.001%-0.10% per weight nanoparticles and water, wherein the nanoparticles carry special cellular water soluble binding groups like carboxyl, amino or phosphonate linkers, amino acids, peptides, antibodies, receptor binding proteins in a formulation that is liquid at 4° C.-32° C. and forms a gel at 37° C. Unlike the use of nanocarriers to enable slow release of drugs the formulation used in this invention is aimed at preventing the nanoparticle from rapid diffusion and keeping their quantum characteristics and properties locally for local action; i.e. only a certain amount of particles will ensure sufficiently strong fluorescence will enable good visibility for a period of days. Furthermore, the present invention refers to the in vivo medical use of the inventive formulation and methods of their preparation.

Another embodiment of the present invention relates to a pharmaceutical formulation for injection comprising 17% to 20% per weight of poloxamer 407, preferably 18% to 19%, more preferably 18% (18% refers to 18.0% to 18.5%) per weight of poloxamer 407, and 3%-15% per weight of poloxamer 188, preferably 3%-7% per weight of poloxamer 188, 0.001%-0.15% per weight of nanoparticles, preferably 0.005%-0.10% per weight, more preferably 0.05%-0.10% per weight of nanoparticles and water or an aqueous buffer. Depending on the nanoparticles also ranges of 0.001%-0.01% and 0.01% to 0.05% per weight of nanoparticles are preferred.

The amount of nanoparticle in the formulation of the present invention differs depending on the characteristics of the nanoparticle used. For example the concentration of Cd/Se quantum dots (0.01-0.03% per weight) is rather small because on the one hand these quantum dots are the brightest one, which means they have a high quantum yield, and on the other hand cadmium is rather toxic. The concentration of Zn/Se quantum dots should and could be 5 to 10 times higher because these quantum dots have a lower quantum yield but are on the other hand zinc less toxic.

Preferably the present invention relates to a pharmaceutical formulation consisting of 0.001 to 14.0% per weight quantum dots, preferably of 0.01 to 10.0% per weight quantum dots, more preferably of 0.10 to 0.5% per weight quantum dots. A particularly preferred formulation consists of 15% per weight poloxamer 407, 15% per weight poloxamer 188, 0.01 to 0.15% per weight quantum dots and up to 100% per weight Aqua ad injectabilia. Another preferred embodiment of the present invention relates to a pharmaceutical formulation consisting of 7% per weight quantum dots, 18% per weight poloxamer 407, 5% per weight poloxamer 188 and 70% per weight Aqua ad injectabilia. Preferred is further a pharmaceutical formulation consisting of 7% per weight quantum dots, 18% per weight poloxamer 407, 10% per weight poloxamer 188 and 65% per weight Aqua ad injectabilia.

The inventors found that not only the kind of poloxamer and the ratio of poloxamer 407 to poloxamer 188 influence the characteristics of the composition and in particular the transition temperature but also the nanoparticles themselves and further additives. Addition of buffers instead of pure water did not change the sol-gel properties of the pharmaceutical formulations. But addition, even of slight amount, of organic solvents, such as hexane and toluene results in instable preparations. Therefore it is preferred that the pharmaceutical formulation of the present invention does not include organic solvents. Thereby organic solvents are carbon based solvents which means that these solvents have carbon atoms present in the structure of their compound.

The pharmaceutical formulation according to the invention is suitable for medical imaging. Medical imaging is the technique and process of creating visual representations of the interior of a body for clinical analysis and medical intervention. The characteristic to be suitable for medical imaging is ensured on one hand by the kind and ratio of the mixture of used poloxamers and on the other hand by the choice of the nanoparticle. It is preferred that the nanoparticles used in the inventive formulation are either fluorescent or magnetic or paramagnetic or both or can in other ways enhance the contrast of structures, tissues or lesions or fluids within the body.

Therefore preferred pharmaceutical formulations according to the invention can be used for imaging technologies selected from the group consisting of X-ray radiography, magnetic resonance imaging, medical ultrasonography or ultrasound, endoscopy, elastography, tactile imaging, thermography, medical photography and nuclear medicine functional imaging techniques as positron emission tomography.

Therefore, a particular preferred embodiment of the present invention relates to a formulation consisting of 2% per weight quantum dots, 18% per weight poloxamer 407, 5% per weight poloxamer 188 and 75% per weight Aqua ad injectabilia. Particularly, preferred is further a formulation consisting of 3.5% per weight quantum dots, 18% per weight poloxamer 407, 10% per weight poloxamer 188 and 68.5% per weight Aqua ad injectabilia.

The term “nanoparticle” or “ultrafine particles” as used herein refers to a small object that behaves as a whole unit with respect to its transport and properties and have a diameter between 1 and 500 nanometers in size, preferably between 1 and 100 nanometers. Thus, in preferred embodiments, the term “nanoparticle” refers to a discrete nano-object where all three Cartesian dimensions are less than 100 nm.

The term “poloxamer” as used herein refers to poly(ethylene glycol)-block-poly(propylene glycol)-block-poly(ethylene glycol), block copolymers of ethylene oxide and propylene oxide falling under the following general formula:

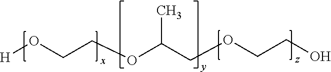

Poloxamers are also known by the trade names Synperonics®, Pluronics®, Lutrol® and Kolliphor®. They are widely used in pharmaceuticals, cosmetics and nutraceuticals and recognized as very safe excipients. Because the lengths of the polymer blocks can be customized, many different poloxamers exist having slightly different properties. The generic term “poloxamer” is commonly followed by three digits, the first two digits×100 give the approximate molecular mass of the polyoxypropylene core, and the last digit×10 gives the percentage polyoxyethylene content (e.g., Poloxamer 407 or P407=Poloxamer with a polyoxypropylene molecular mass of 4,000 g/mol and a 70% polyoxyethylene content). It is preferred that poloxamer 407 is used in combination with poloxamer 188. In regard to poloxamer 407, x and z in the general formula have the value of 101 and y is 56, wherein for poloxamer 188 x and z have the value of 80 and y is 27.

Preferably the present invention relates to a pharmaceutical formulation, wherein the nanoparticles optionally coupled with targeting ligands are selected from the group comprising or consisting of: semiconductor nanoparticles, quantum dots (of different metal content), dye-doped silica nanoparticles, preferably fluorescent dye-doped silica nanoparticles, dye dope calcium particles (coupled with or without targeting ligands), dye labeled liposomes, fluorescent carbon nanoparticles, iron oxide nanoparticles eg called SPION), gold nanoparticles, gold nanorods, gold nanoshells (silica particles coated with a nanometer-thick gold shell), nanoparticles containing organic chromophores like indocyanine green (ICG), and silver/organic dye composites nanoparticles, nanoparticles containing a gadolinium oxide core and combinations of the afore-mentioned nanoparticles. The nanoparticles and preferably the fluorescent nanoparticles used within the present invention are preferably functionalized or derivatized with targeting ligands and especially with targeting ligands for cancer cells.

Suitable dye-doped silica nanoparticles and in particular fluorescent dye-doped silica nanoparticles and methods for their preparation are for example disclosed in WO2011/109214 A2 and in WO2010/030119 A2. Dye labeled, fluorescent liposomes can be purchased, for example from FormuMax Scientific, USA. Fluorescent carbon nanoparticles which can be formulated as described herein, and their synthesis has been described by Susanta Kumar Bhunia et al. (Carbon Nanoparticle-based Fluorescent Bioimaging Probes; Sci Rep. 2013; 3: 1473).

Suitable nanoparticles and in particular fluorescent nanoparticles comprising an inorganic core and a passivation layer containing imidazole as well as methods for their preparation are disclosed in WO 2007/057182 A2.

It is particularly preferred that fluorescent nanoparticles of the pharmaceutical formulation according to the invention are quantum dots. The term “quantum dots” as used herein refers to nanocrystals made of semiconductor materials with diameters in the range of 2-10 nanometers. It is important that the diameters are small enough to exhibit quantum mechanical properties. The term nanocrystals refers to nanometer-sized single crystals, or single-domain ultrafine particles. Preferred quantum dots are nanometer-scale (roughly protein-sized) atom clusters, containing from a few hundred to a few thousand atoms of a semiconductor material (such as cadmium with selenium or tellurium), which has been coated with an additional semiconductor shell (such as zinc sulfide) to improve the optical properties of the material.

Therefore, one preferred embodiment of the present invention relates to pharmaceutical formulations, wherein the quantum dots contain a core made of cadmium selenide or cadmium telluride which preferably are coated with zinc sulfide. Other suitable quantum dots may be selected from the group comprising or consisting of:

InP/ZnS core/shell quantum dots, Mn/ZnSe quantum dots, Mn doped ZnO quantum dots, CuInS/ZnS core/shell quantum dots, Gold NanoRods, ZnCuInS/ZnS core/shell quantum dots, ZnSe/ZnS CuInS/ZnS core/shell quantum dots, CuInS/ZnS core/shell quantum dots and InP/ZnS quantum dots. In general, many different kinds of quantum dots can be purchased, for example from life technolgies, mkNANO or Mesolight. The quantum dots are used in the pharmaceutical formulation in an amount of 0.001%-0.15% per weight of at least one quantum dot, preferably 0.01%-0.10% per weight, more preferably 0.015%-0.075% per weight of at least one quantum dot. The term “at least one quantum dot” refers to at least one sort of quantum dots and thus comprises also mixtures of different sorts of quantum dots. A preferred quantum dot is a quantum dot having an emission in the visible range (350-700 nm), i.e. a sharp emission peak beyond tissue protein auto-fluorescence, for instance a maximum of about 655 nm, such as QDot® 655.

Preferred as fluorescent nanoparticles are nanocrystals containing a core made of atoms of a semiconductor material, which has been coated with an additional semiconductor shell. Preferred are further pharmaceutical formulations according to the invention, wherein the nanocrystals contain a core of cadmium selenide or telluride and or zinc selenide and are coated with zinc sulfide.

The present invention especially relates to pharmaceutical formulations containing nanoparticles which are linked to biomolecules. Thus, one aspect of the present invention is directed to a nanoparticle (being e.g. fluorescent or ferromagnetic) containing molecular and biological binding groups. These biomolecules or biogenic substances include large macromolecules such as proteins, polysaccharides, lipids, antibodies, and nucleic acids, as well as small molecules such as primary metabolites, secondary metabolites, peptides, binding (but otherwise inactive) antibodies and natural products. However these biomolecules have no therapeutically effect, at least not when bound to the nanoparticle and are not released in vivo. These bioconjugates (nanoparticle linked or conjugated to biomolecules) or targeted nanoparticles are suitable for specific or controlled staining of a cell type (such as cancer cells) or a specific tissue or substructures (specific molecules in the cell membrane or specific structures in a tissue). The present invention relates further to pharmaceutical formulations, wherein the nanoparticles contain a ligand or targeting ligand selected from the group consisting of peptides, antibodies, si-RNA (small interfering RNA), spiegelmers, and oligonucleotides. Suitable target molecules or ligands which can be coupled and preferably covalently coupled to the nanoparticles are biologically active structures, e.g. protein receptors, ligands, enzymes, substrates, antibodies, antigens, chelators and the like. The ligands of the targeted nanoparticles can for example specifically interact with tumor epitopes and would therefore allow marking tumors to assess the spread of tumors or their metastases and single cell flotation. A preferred biomolecule would be an adhesin as disclosed in WO 2009/106102 A1. The present invention especially relates to pharmaceutical formulations containing nanoparticles, such as quantum dots, which are linked to dipeptides and particularly to dipeptides made of histidine (histidine-histidine—dipeptides).

The present invention relates also to pharmaceutical formulations containing nanoparticles which are linked to polyethylenglycol (PEGylated nanoparticles). One embodiment of the present invention relates to a pharmaceutical formulation according to the invention, wherein the solvent is water. Another embodiment of the present invention relates to a pharmaceutical formulation according to the invention, wherein the solvent is an aqueous buffer. Preferred aqueous buffers are phosphate buffer (PP) or phosphate buffered saline. Phosphate buffers are based on salts of phosphoric acid. Phosphoric acid has multiple dissociation constants, therefore, one can prepare phosphate buffers with varies pH values. The buffer is most commonly prepared using dihydrogen phosphate and dibasic monohydrogen phosphate. One can for example use monosodium phosphate and its conjugate base, disodium phosphate. Also potassium phosphates are commonly used. Phosphate buffered saline (PBS) is a water-based salt solution containing sodium phosphate, and potassium chloride and potassium phosphate. PBS is often used in biology and pharmaceutical formulations because it is isotonic and non-toxic to cells. Preferred buffering agents are monopotassium phosphate, citric acid, acetic acid, phosphoric acid, boric acid, triethanolamine, N-cyclohexyl-2-aminoethanesulfonic acid (CHES), tris(hydroxymethyl)aminomethane (TRIS), 4-(2-hydroxyethyl)-1-piperazineethanesulfonic acid (HEPES), piperazine-N,N′-bis(2-ethanesulfonic acid) (PIPES) and 3-(N-morpholino)propanesulfonic acid (MOPS).

Therefore preferred embodiments refer to pharmaceutical formulation according to the invention comprising further at least one compound of the group comprising or consisting of monohydrogen phosphate, chloride, dihydrogen phosphate, tris(hydroxymethyl)aminomethane, citric acid monohydrate, trisodium citrate dehydrate, acetate trihydrate, glycine, bicarbonate, ammonia and borate.

The pharmaceutical formulation according to the present invention can effectively be used for optimal endoscopic or coloscopic application and laparoscopic visualization, as well as injected into tissue from external surfaces or deep injection, because it has the following characteristics:
Liquid at temperatures from 4° C. to 32° C., incl. room temperature.
Liquid during endoscopic procedure and filling of the injection device.
Injectable by an injection device, i.e. endoscopy capillary
Becoming semisolid or gel-like at body temperature (within 30 minutes)
Adequate stability and mechanical properties after injection in the tissue
stable at pH of 7.0 to 8.6 (particularly at physiological pH values)
Excipients of pharmaceutical grade quality, approved for injections in humans
Excipients of non-animal origin

Therefore, another embodiment of the present invention relates to the pharmaceutical formulations according to the present invention for use pre-operatively, in particular in the marking or staining of specific areas in the patient's body. The pharmaceutical formulations according to the present invention are particularly suitable for use as a marker of bodily areas applied by injection directly into an organ or diseased body region and further for use as a marker of tumor areas. Marking of tumor areas and the tumour tissue specifically enables surgical precision of tumour removal.

The pharmaceutical formulations according to the present invention are especially developed for use in preoperative staging of bodily areas and especially gastrointestinal areas applied by injection during an endoscopic procedure. The pharmaceutical formulations disclosed herein are used for different type of inspections into body spaces like bronchoscopy, otolayngoscopy, cystoscopy, colonoscopy, etc. as well as in open surgery and marking of specific locations for the purpose of visually guided surgery, incl. distal biosensor endoscopy imaging, as well as fiducial markers for computer aided surgery. The newest surgery machines require such markers for orientation purposes especially when the patient moves during surgery.

Besides the effect that the pharmaceutical formulations according to the present invention remain at the site of injection for a prolonged time (typically some days), this enables to mark the lesion in the intestine or colon days before the operation and makes it easier to visually find this lesion until and during surgery. In addition when using nanoparticles containing heavy metal or toxic nanoparticles it is preferred that as many nanoparticles as possible remain at the injection site/marked area to exert their physical marker signal, and because with tumor excision the marked area (lesion; tumor tissue) and also the nanoparticles are removed from the patient's body and none or less unwanted systemic exposure will take place.

The use of poloxamers in the inventive formulation has advantages: they favourably bind to cell membranes, as has been shown with the inventive formulation in binding experiments at human tumour tissue slices. Nanoparticles without special binding groups, like Quantum dots do not themselves bind to cell membranes but the poloxamers mediate binding to cell membranes.

Alternatively, the pharmaceutical formulations according to the present invention are suitable for use as a marker of areas in the skin, preferably in the dermis of skin and/or in mucosal layers. The inventors found that it is preferred that in the pharmaceutical formulation according to the invention, the nanoparticles are not integrated in the self-forming micelles, which are generated by the poloxamers after dissolution in water. Such poloxamer formulations have been previously described for many water insoluble compounds. However, the pharmaceutical formulation of the present invention contains nanoparticles being dispersed between the micelles formed by poloxamers. This means in case the inventive pharmaceutical formulation is liquid both the poloxamer micelles and the nanoparticles are dispersed in the water or the aqueous buffer and in case of semi-solid or gel-like state the nanoparticles are dispersed in the gel matrix build by the poloxamers. It is especially preferred if the nanoparticles used within the inventive pharmaceutical formulation are hydrophilic.

Further embodiment of the present invention relates to a method for the preparation of the pharmaceutical formulation according to the present invention comprising or consisting of the following steps:
a) dissolving at least one poloxamer, and preferably 17% to 20% per weight of poloxamer 407 and 3% to 15% per weight of poloxamer 188, in water or in an aqueous buffer;
b) stirring for at least 20 hours at 4° C.; and
c) adding at least one nanoparticle.

The term “at least one poloxamer” obviously refers to at least one sort of poloxamer and not a single poloxamer molecule. Analogously, the term “at least one nanoparticle” refers to at least one kind of nanoparticles (preferred are fluorescent and/or paramagnetic nanoparticles) and not a single nanoparticle molecule.

A preferred embodiment of the present invention relates to a method for the preparation of a pharmaceutical formulation comprising or consisting of the following steps:
a) dissolving at least one poloxamer, and preferably 17% to 20% per weight of poloxamer 407 and 3% to 15% per weight of poloxamer 188, in water being precooled to 4° C.-8° C. or in an aqueous buffer being precooled to 4° C.-8° C.;
b) stirring for at least 20 hours at 4° C.; and
c) adding at least one nanoparticle.

Another preferred embodiment of the present invention relates to a method for the preparation of a pharmaceutical formulation comprising or consisting of the following steps:
a) heating up at least one poloxamer, and preferably 17% to 20% per weight of poloxamer 407 and 3% to 15% per weight of poloxamer 188, to a temperature between 70° C.-80° C.;
b) dissolving the melted poloxamer in water heated up to a temperature between 70° C.-80° C. or in an aqueous buffer heated up to a temperature between 70° C.-80° C.;
c) cooling down the obtained mixture to 4° C. while stirring the obtained mixture;
d) stirring for at least 20 hours at 4° C.; and
e) adding at least one fluorescent nanoparticle.

This method involves the mixing under stirring of an aqueous buffer or water which is heated to a temperature within the temperature range of 70° C. to 80° C. with at least one (sort of) poloxamer which is also heated to a temperature within the temperature range of 70° C. to 80° C. Thereafter the obtained mixture is preferably allowed to cool to room temperature under stirring. When room temperature is reached, active cooing, for instance by a cooling bath with ice water, is applied and the obtained mixture is cooled to 4° C. and stirred at 4° C. for at least 20 hours.

Another aspect the present invention refers to pharmaceutical formulations obtainable according to one of the above described methods. These methods ensure that the at least one poloxamer generates micelles and that the added fluorescent nanoparticles are not included into these micelles but are finely dispersed in the pharmaceutical formulation (between the micelles formed by poloxamers) or respectively in the gel-matrix at higher temperatures.

The pharmaceutical formulation for injection or pharmaceutical composition for injection according to the present invention comprises 17% to 20% per weight of poloxamer 407, preferably 18% to 19%, more preferably 18% (18% refers to 18.0% to 18.5%) per weight of poloxamer 407, and 3%-15% per weight poloxamer 188, preferably 3%-7% per weight of poloxamer 188, more preferably 5% of poloxamer 188, 0.001%-0.15% per weight of at least one quantum dot, preferably 0.01%-0.10% per weight, more preferably 0.015%-0.075% per weight of fluorescent nanoparticles and water or an aqueous buffer and will typically be administered by injection. The pharmaceutical formulations may be administered together with further excipients selected with respect to the intended form of administration and consistent with conventional pharmaceutical practices.

Where necessary, the pharmaceutical formulation may also include a solubilizing agent and a local anesthetic to ease pain at the site of the injection. For liquid compositions, especially in the form of solutions for parenteral injections, the pharmaceutical formulation of the present invention may be combined with additives for preservation, for adjusting pH or osmolarity, and/or for providing stability or long term stability. Nevertheless, the inventors could show that additive diminishes fluorescence of the pharmaceutical formulation. Therefore, it is preferred that the pharmaceutical formulation according to the present invention consists of poloxamer, at least one fluorescent nanoparticle and water or an aqueous buffer and in particular consists of 17% to 20% per weight of poloxamer 407, preferably 18% to 19%, more preferably 18% (18% refers to 18.0% to 18.5%) per weight of poloxamer 407, and 3%-15% per weight of poloxamer 188, preferably 3%-7% per weight of poloxamer 188, more preferably 5% of poloxamer 188, 0.001%-0.15% per weight of at least one quantum dot, preferably 0.01%-0.10% per weight, more preferably 0.015%-0.075% per weight of fluorescent nanoparticles and water or an aqueous buffer. It is particularly preferred that the pharmaceutical formulation according to the invention does not contain D,L alpha-tocopherol.

Pharmaceutical compositions for injection are typically sterile solutions. Therefore it is preferred that the aqueous portion of the pharmaceutical formulation is endotoxin free water such as highly purified water or water for the purpose of injection (Aqua ad injectabilia) or is based on highly purified water or water for the purpose of injection (Aqua ad injectabilia). Furthermore the pharmaceutical formulation may be sterilized using commonly known methods, such as methods including ionizing radiation (gamma and electron-beam radiation), and gas (ethylene oxide, formaldehyde).

Different sugars such as saccharose or trehalose may be added as cryoprotecting agents or for adjusting osmolarity of the solution. Polyalcohols, for example mannitol and sorbitol, may be used as stabilizer or for adjusting osmolarity.

Furthermore the composition may contain amino acids, such as glycine, arginine, leucine, carnosine or proline, as stabilizer. Further stabilizer of the solution may be urea or human serum albumin.

Benzyl alcohol may be used as a bacteriostatic preservative at low concentration in the composition and especially in liquid form preparations of the present invention. In addition antioxidants, such as methionine, may be used.

### DESCRIPTION OF THE FIGURES

FIG. 1 shows a standard curve of fluorescence (A) in regard to different amounts of investigated, inventive pharmaceutical formulation determined by fluorimetric measurement and a standard curve for cadmium determination (B).

FIG. 2 shows quantum dot concentrations on the apical side and in the cellular layer after 1 h (A) and 3 h (B) incubation. All quantum dot concentrations on basal sides were below quantification level. Therefore these values are not displayed.

FIG. 3 shows cadmium concentrations on the apical side and in the cellular layer after 1 h (A) and 3 h (B) incubation. Cadmium concentrations on basal sides were below quantification level. Therefore these values are not displayed.

FIG. 4 shows characteristics of inflammatory reactions of mild, moderate and severe intensity in the skin.

FIG. 5 shows shear-dependent viscosity behavior of Gel-F4 (A), Gel-F5 (B), QDot 655 F4 (3.5%; C) and QDot F5 (7%; D) (results of example 11).

FIG. 6: Dose and time dependency of formulation F7. The various QDot® doses were applied topical and intradermal onto the porcine colon and subsequently measured using the Maestro system on stage 1 using the blue filter. Values for measurements using 200 ms exposure time are shown.

FIG. 7: Dose and time dependency of formulation F7. The various QDot® doses were applied topical and intradermal onto the porcine colon and subsequently measured using the Maestro system on stage 1 using the UV filter. Values for measurements using 200 ms exposure time are shown.

FIG. 8: Inventive preparations containing 3.5% or 1% of CANdots Series M12, respectively or 3.5% or 10% of [ZrO]2+[MFP]2 in semi-solid state on day 8 after preparation and 7 days of storage at 37° C. The following examples are included to demonstrate preferred embodiments of the invention. It is to be understood that the forms of the invention shown and described herein are to be taken as examples of embodiments. However, those of skill in the art should, in light of the present disclosure, appreciate that many changes can be made in the specific embodiments which are described by the examples and still obtain a like or similar result without departing from the spirit and scope of the invention.

### EXAMPLES

### Example 1: Preparation of an Inventive Pharmaceutical Formulation

150 mg of poloxamer 407 (Lutrol®F127 from BASF; Germany) and 150 mg poloxamer 188 (Lutro®F68 from BASF; Germany) were mixed together with 20 mg DL-α-Tocopherol (Hanseler) and this mixture was added to 610.2 mg pre-cooled water (Aqua ad injectabilia from BBraun Melsungen; approx. 5° C.) in a transparent glass beaker and constant agitation with a magnetic stirring bar was kept for 24-48 hours at 4° C.

Subsequently, 69.8 mg of Qdot®655 solution (Qdot®655 dipeptide (His-His) quantum dots *commercial use*; Lot Number: 812401-1 and 812401-2, in 50 mM borate buffer, pH 8.3 from life technologies brand of Thermo Fisher Scientific, USA) was added to 930.2 mg of the pharmaceutical formulation and mixed to homogeneity at room temperature. Resulting pharmaceutical formulations were stored at 4° C. (2° C. to 8° C.) under light protected conditions.

### Example 2: Preparation of an Inventive Pharmaceutical Formulation

180 mg of poloxamer 407 (Lutrol®F127 from BASF; Germany) and 50 mg poloxamer 188 (Lutrol®F68 from BASF; Germany) was added to 700.2 mg pre-cooled water (Aqua ad injectabilia from BBraun Melsungen; approx. 5° C.) in a transparent glass beaker and constant agitation with a magnetic stirring bar was kept for 24-48 hours at 4° C.

Subsequently, 69.8 mg of Qdot®655 solution (Qdot®655 dipeptide quantum dots *commercial use*; Lot Number: 812401-1 and 812401-2, in 50 mM borate buffer, pH 8.3 from life technologies) was added to 930.2 mg of the pharmaceutical formulation and mixed to homogeneity at room temperature. Resulting pharmaceutical formulations were stored at 4° C. (2° C. to 8° C.) under light protected conditions.

### Example 3: Preparation of an Inventive Pharmaceutical Formulation

180 mg of poloxamer 407 (Lutrol®F127 from BASF; Germany) and 100 mg poloxamer 188 (Lutrol®F68 from BASF; Germany) was added to 650.2 mg pre-cooled water (Aqua ad injectabilia from BBraun Melsungen; approx. 5° C.) in a transparent glass beaker and constant agitation with a magnetic stirring bar was kept for 24-48 hours at 4° C.

Subsequently, 69.8 mg of Qdot®655 solution (Qdot®655 dipeptide quantum dots *commercial use*; Lot Number: 812401-1 and 812401-2, in 50 mM borate buffer, pH 8.3 from life technologies) was added to 930.2 mg of the pharmaceutical formulation and mixed to homogeneity at room temperature. Resulting pharmaceutical formulations were stored at 4° C. (2° C. to 8° C.) under light protected conditions.

### Example 4: Preparation of an Inventive Formulation Using “Hot Process”

180 mg of poloxamer 407 (Lutrol®F127 from BASF; Germany) and 100 mg poloxamer 188 (Lutrol®F68 from BASF; Germany) was weighted out in a glass tube and heated up to 70° C.-80° C. Aqua ad injectabilia (BBraun Melsungen) was heated up to 70° C.-80° C. and mixed with the melted poloxamers. Constant agitation with a magnetic stirring bar was kept for 24-48 hours at 4° C.

Subsequently, 69.8 mg of Qdot®655 solution (Qdot®655 dipeptide quantum dots *commercial use*; Lot Number: 812401-1 and 812401-2, in 50 mM borate buffer, pH 8.3 from life technologies) was added to 930.2 mg of the pharmaceutical formulation and mixed to homogeneity at room temperature. Resulting pharmaceutical formulations were stored at 4° C. (2° C. to 8° C.) under light protected conditions.

### Example 5: Preparation of an Inventive Pharmaceutical Formulation with PP

0.1M Phosphat buffer pH 7.4 were prepared: 6.805 g of potassium dihydrogen phosphate were dissolved in aqua ad injectabilia to obtain 500 ml of 0.1M stock solution A. 8.90 g of disodium hydrogenphosphate dihydrate were dissolved at room temperature in aqua ad injectabilia to obtain 500 ml Stock solution B. 81.8 ml stock solution B and 18.2 ml stock solution A were mixed to give a final volume of 100 ml 0.1 M phosphate buffer, pH 7.4. After sterile filtration (0.22 μm) buffer was stored at 4° C.

180 mg of poloxamer 407 (Lutrol®F127 from BASF; Germany) and 50 mg poloxamer 188 (Lutrol®F68 from BASF; Germany) was weighted out in a glass tube and heated up to 70° C.-80° C. Phosphate buffer (0.1M, pH 7.4) was heated up to 70° C.-80° C. and mixed with the melted poloxamers up to 930.2 mg. Constant agitation with a magnetic stirring bar was kept for 24-48 hours at 4° C.

Subsequently, 69.8 mg of Qdot®655 solution (Qdot®655 dipeptide quantum dots *commercial use*; Lot Number: 812401-1 and 812401-2, in 50 mM borate buffer, pH 8.3 from life technologies) was added to 930.2 mg of the pharmaceutical formulation and mixed to homogeneity at room temperature. Resulting pharmaceutical formulations were stored at 4° C. (2° C. to 8° C.) under light protected conditions.

### Example 6: Preparation of an Inventive Pharmaceutical Formulation with PBS

180 mg of poloxamer 407 (Lutrol®F127 from BASF; Germany) and 50 mg poloxamer 188 (Lutrol®F68 from BASF; Germany) was weighted out in a glass tube and heated up to 70° C.-80° C. Phosphate buffered saline (PBS; 12 mM phosphate, 137 mM NaCl and 2.70 mM KCl, pH 7.4) was heated up to 70° C.-80° C. and mixed with the melted poloxamers up to 930.2 mg. Constant agitation with a magnetic stirring bar was kept for 24-48 hours at 4° C.

Subsequently, 69.8 mg of Qdot®655 solution (Qdot®655 dipeptide quantum dots *commercial use*; Lot Number: 812401-1 and 812401-2, in 50 mM borate buffer, pH 8.3 from life technologies) was added to 930.2 mg of the pharmaceutical formulation and mixed to homogeneity at room temperature. Resulting pharmaceutical formulations were stored at 4° C. (2° C. to 8° C.) under light protected conditions.

### Example 7: Evaluation of Physical Formulation Properties

### A: Appearance and pH Value

The appearance of the pharmaceutical formulations was assessed by visual observation: homogeneity, phase separation. Furthermore, color was assessed in quantum dots containing formulations. pH values of the pharmaceutical formulations (after 1:10 w/v dilution with distilled water) was detected using a 766 Knick pH meter.

### B: Visual Detection of Formulation Viscosity

A simple test tube inverting method was used to determine formulation viscosity. 5 ml of the respective pharmaceutical formulation consisting of different amounts of poloxamers (w/o additional excipients) were transferred to glass vials sealed with lamellar plugs and placed in a thermostat (4° C.) water bath. Temperature was stepwise increased up to 37° C. and left to equilibrate for 10 minutes at each new setting. At predefined temperatures viscosity was visually detected by turning the glass tubes upside down and classifying the flow properties of the transparent pharmaceutical formulation into four categories:

1: liquid like water
2: slightly viscous
3: viscous to highly viscous
4: semisolid

This method is easy to perform but shows limitations: Exact detection of gelation temperature is not possible as sol-gel transition occurs within a large temperature interval which is influenced by experimental set-up.

### C: Injectability of Formulations at Room Temperature

Injectability of formulations was assessed by aspiration of 1 ml of the respective pharmaceutical formulation into a 1 ml syringe to which a 0.9×40 mm cannula was attached. The force necessary to fill the syringe through the cannula was assessed as

1: force comparable to aspiration of water
2: force comparable to aspiration of oil
3: strong forces required

### D: Measurement of Gel Strength

Gel strength of selected formulations was assessed by the following procedure: 1 ml g of the respective pharmaceutical formulation was poured into a 2 ml screw capped glass vial and gelation was induced by exposure of the pharmaceutical formulation to 37° C. Gel formation was observed within several minutes, usually within 1 to 10 minutes. To ensure full gel formation samples were incubated at 37° C. for approx. 30 minutes.

After gel formation 1 ml of prewarmed water (37° C.) was added, the vials screwed again and incubated in the water bath with gentle shaking at 37° C. (30 rpm). The gel strength was determined as the time necessary for dissolution of the gel.

### Results

In a first series of experiments “empty” hydrogels i.e. hydrogels without addition of Qdots®655 were tested with regard to their thermo sensitive behavior, transparency and pH value. Amount of Qdot®655 was substituted by.

Empty gels in concentrations of 15%, 20%, 25% and 30% of Poloxamer 407 were prepared analogue to example 1.

Pharmaceutical formulations containing 15% of Poloxamer 407 appeared liquid or slightly viscous, respectively, at temperatures between 4° C. and 22° C. Gel strength increased with temperature: at 30° C. and still at 32° C. formulations appeared viscous and became semisolid at 37° C. Formulations consisting of 20% or 25% of Poloxamer 407 were liquid at 4° C. and became semisolid at room temperature; a hydrogel prepared with 30% of Poloxamer 407 was already highly viscous at 4° C.

Poloxamer 407 formulations appeared transparent and colorless; they were homogeneous after 24 to 48 hours at temperatures of approx. 4° C.

### Effect of Various Poloxamer 407/188 Ratios:

Properties of pharmaceutical formulations containing Poloxamer 407 as main excipient, such as thermo sensitive behavior of pharmaceutical formulations, can be modified in the presence of different additives. Poloxamer 188 in different concentrations (5%, 10%, 15%, 20%) was added to Poloxamer 407 (15%, 20%, 25%, 30%) and the resulting pharmaceutical formulations assessed as to their physical properties. Addition of Poloxamer 188 led to change of gel formation:

Pharmaceutical formulations composed of 15% Poloxamer 407 and 5% to 15% of Poloxamer 188 appeared viscous at room temperature, but did not achieve the semisolid state at 37° C. This could be achieved only by addition of Poloxamer 188 at higher concentrations than 15%. Pharmaceutical formulations consisting of 20%, 25% or 30% of Poloxamer 407 were solid at 37° C.; however, viscosity at lower temperatures was high as well.

Pharmaceutical formulations composed of 15% or 20% Poloxamer 407 showed the best physical properties according to thermo sensitive behavior. Thus, gels were tested using 18% of Poloxamer 407 in combination with different concentrations of Poloxamer 188 (5%, 10%, 15%, 18%. Pharmaceutical formulations with 18% Poloxamer 407 and either 5% or 10% of Poloxamer 188 were liquid at 4° C., slightly viscous at 22° C. and semisolid at 37° C. These compositions showed the required thermo sensitive characteristics. Viscosity at 4° C. and room temperature increased in presence of higher Poloxamer 188 concentrations.

### Influence of Phosphate Buffers on Poloxamer 407/188 Preparations

The influence of buffers on Poloxamer 407/188 formulations was tested by preparing different pharmaceutical formulations with either phosphate buffer (PP, 0.1M, pH 7.4) or phosphate buffered saline (PBS, 0.1M, pH 7.4) as aqueous phase.

Addition of buffers instead of pure water did not change the viscous properties of the pharmaceutical formulations.

### Influence of DL-α-Tocopherol on Poloxamer 407/188 Preparations

DL-α-Tocopherol is known to reduce the critical micelle concentration and is expected to stabilize pharmaceutical formulations according to the invention. DL-α-Tocopherol was added to melted Poloxamer 407/188 mass previous to the addition of water.

Poloxamer 407/188 formulations were prepared with DL-α-Tocopherol in different concentrations: 0.5%, 1.0%, 1.5% or 2.0% according to example 1.

Pharmaceutical formulations containing DL-α-Tocopherol appeared transparent and slightly yellowish in dependence of the tocopherol concentration.

DL-α-Tocopherol in pharmaceutical formulations composed of 15% Poloxamer 407 and 5% or 10% Poloxamer 188 appeared transparent and homogeneous. The fluorescence properties are reduced as compared to identical pharmaceutical formulations without D,L-α-Tocopherol.

### Nanoparticle Containing Pharmaceutical Formulations

After selecting most promising “empty” formulations (composed of 15% Poloxamer 407 and 5% or 10% Poloxamer 188) based on their thermo sensitive properties, the same preparations were loaded with Qdot®655. Qdots appeared evenly spread; no turbidity or sedimentation could be observed even after two months of storage at 4° C.

### Injectability of Nanoparticle Containing Formulations at Room Temperature

Pharmaceutical formulations according to the invention have to be liquid or slightly viscous at room temperature to allow injection by means of suitable injection devices attached to a laparoscope. To test the pharmaceutical formulations for this property, the force necessary to fill the syringe through a cannula was assessed. All selected pharmaceutical formulations could by aspirated through the 0.9×40 mm needle into the syringe.

### Dissolution of Gels—Gel Strength

Selected pharmaceutical formulations were investigated for their gel strength as described above. Dissolution of gels in water over time was taken as an indicator for stability of gels after injection into tissue.

The results indicated that 15% of Poloxamer 407 alone could not provide suitable gel stability for the intended application because it loses its gel properties within minutes after overlay with water.

Pharmaceutical formulation containing 15% Poloxamer 407 and 20% Poloxamer 188 started dissolving after 30 minutes of incubation.

Quantum dot loaded formulations composed of 18% to 20% of Poloxamer 407 and 5% to 15% of Poloxamer 188 were the most stable under the experimental conditions described: phase separation between gel and water phase was clearly visible over hours.

### pH Values of Gels

pH values were measured in water diluted test samples. pH values of “empty” gels range between 6.84 and 7.58 depending on the composition of the samples. As expected addition of Qdot®655 (in its original borate buffer) led to shift of pH values to 7.03 to 8.38. This is due to the high pH of borate buffer (50 mM pH 8.3)

### Discussion and Conclusion

The aim of this study was to prepare pharmaceutical formulations containing fluorescent nanoparticles having thermo-responsive gelation behavior to be used effectively as injectable formulation for sustained marking of selected tissue areas. Poloxamer based hydrogels were shown to meet these requirements. Poloxamer 407 and 188 have been reported to be the least toxic of commercially available copolymers and is approved for parenteral application.

Viscosity and injectability of 15% Poloxamer 407 formulations were shown to fulfill the necessary requirements. However, Poloxamer 407 as single excipient rapidly mixes with water and gel erosion is observed; this would lead to rapid disappearance of fluorescence signal after local injection into tissue. Stability of gels was improved by addition of Poloxamer 188.

Pharmaceutical formulations composed of 18-20% Poloxamer 407/5% to 10% Poloxamer 188/70% or 65% of water or aqueous buffer are slightly viscous at refrigerated temperatures, viscous at room temperature and semisolid at body temperature. Aspiration into syringes and injection of the pharmaceutical formulation into tissue was easy to be performed.

D,L-α-Tocopherol was added to the optimized pharmaceutical formulation since in experience of the inventors formulations with this excipient are stabilized especially when diluted. D,L-α-Tocopherol changes the color of the Qdot®655 preparations and slightly increases viscosity at all temperatures. The fluorescence properties are reduced as compared to identical pharmaceutical formulations without D,L-α-Tocopherol.

### Example 8: Permeability of CACO-2 Cells for Quantum Dots in an Inventive Formulation

The aim of the study was to investigate the permeability of four different pharmaceutical formulations according to the invention across Caco-2 cell monolayer assay. Membrane transport was measured in the apical-to-basolateral (A2B) direction.

### Tested Formulations:

Control formulation (BB): 300 μg QDot®655 solution dissolved in 4651 μL borate buffer, (Qdot®655 dipeptide quantum dots *commercial use*; Lot Number: 812401-1 and 812401-2, in 50 mM borate buffer, pH 8.3 from life technologies)
Formulation 1: 300 μg QDot® 655 (Qdot®655 dipeptide quantum dots) plus 4651 μL consisting of 15% poloxamer 407/15% poloxamer 188/2% DL-α-Tocopherol/61% Aqua ad injectabilia.
Formulation 2: 300 μg QDot® 655 (Qdot®655 dipeptide quantum dots) plus 4651 μL consisting of 18 poloxamer 407/5% poloxamer 188/70% Aqua ad injectabilia.
Formulation 3: 300 μg QDot®655 (Qdot®655 dipeptide quantum dots) plus 4651 μL consisting of 18% poloxamer 407/10% poloxamer 188/65% Aqua ad injectabilia.

### Cell Preparation

One vial of Caco-2 cells (ECACC, 09042001), stored in liquid nitrogen, was thawed and seeded in Caco-2 cell media (500 mL DMEM+10% FBS+1% L-Alanyl-L-Glutamine+1% antibiotic/antimycotic+1% MEM NEAA) in a 75 cm² flask. Cells were passaged two times per week. In order to create a cell monolayer, 2×10⁵Caco-2 cells (passage 22) were seeded on top of every 24-well plate insert (Millicell 24-well plates, Millipore) in 0.4 mL of Caco-2 media and 26 mL media was added to the bottom compartment. Cells were incubated for 21 days in a CO² incubator with replacement of media every three days.

### Dispersion of Quantum Dot Formulations on Caco-2 Cell Monolayers

Dispersion of QDot®655 formulations in contact with the Caco-2 cells and medium in the well was investigated using photography under UV light. One 24-well plate without cells was used to optimize camera settings (Camera Canon EOS 350D, lens Tamron 28-75 f2.8, excitation UV lamp 365 nm). 20 μL of each pharmaceutical formulation, or control beads (Fluoresbrite Plain YG 60 micron Microspheres, Polysciences), was added with or without cell culture medium and camera settings were adjusted accordingly. Prior to start of the experiment, the medium was replaced by fresh medium and cells were left to equilibrate for one hour at 37° C., 5% CO2 (incubator). Different volumes (10, 20 and 40 μL) of each QDot®655 formulation were added to the apical side of the Caco-2 monolayer in cell medium. In parallel, 20 μL of each pharmaceutical formulation was added to cells without medium to enhance the signal. The plate was captured by photography at three time points: 0, 30 and 60 minutes.

### Permeability of Quantum Dot Formulations Through the Caco-2 Monolayer

Prior to start of the experiment, monolayer integrity was inspected by transepithelial resistance (TEER) measurement, using Millicell ERS Volt-Ohm Meter, according to the manufacturer's instructions. Cells were used for further permeability testing only if the Caco-2 cell monolayer showed good consistency (TEER 1500 Ohm/cm²).

20 μL of each QDot®655 formulation were added to the apical side of the Caco-2 cell monolayer (in 0.4 mL of medium) in triplicates. Two cell plates were used, one of which was incubated for 1 h and the other for 3 h. Following addition of QDot®655, the plates were incubated on a Vibrating Titramax 1000 Shaker at 37° C. In parallel, 20 μL of QDot®655 were mixed with 400 μL of medium (final concentration 15 ng/μL) and incubated together with cell plates to be used for standard curve preparation. Following 1 h and 3 h of incubation, medium from the apical and basal side (separately) of the cell monolayer from each plate was transferred into two aliquots—one for fluorescence measurement and one for Cd determination (100 μL each). Fluorescence was measured immediately as described below, and samples for Cd determination were kept frozen at −20° C. until analysis.

At both time points, for the purpose of determination of cell-associated concentrations, cells were washed two times with sterile PBS and lysed with 200 μL 0.5% Triton X-100, resuspended by pipette and diluted with an additional 200 μL 0.5% Triton X-100. The lysate was divided into two separate aliquots for fluorescence measurement and Cd determination (100 μL each).

### Fluorescence Measurement

The standard curve for each pharmaceutical formulation was generated from five-fold dilution steps, five points (1×, 5×, 25×, 125×, 625×), starting from 15 ng/μL. Pure medium was used as blank.

For each sample, 100 μL of medium from the apical and basal side, as well as cell lysate, was added into a well of a black 96-well plate, and fluorescence was measured at 340 nm (exc) and 665 nm (ems) on an EnVision 2104 (Perkin Elmer) reader.

### Determination of Cadmium Concentration

The determination of Cd was performed at the Institute for Medical Research and Occupational Health, Analytical Toxicology and Mineral and Analytical and Toxicology and Mineral and Metabolism Unit (under the supervision of Dr. Sc. Jasna Jurasović, Head of the Analytical Toxicology and Mineral Metabolism Unit).

### Instruments and Reagents

Sample decomposition was performed using an UltraCLAVE IV microwave digestion system (Milestone S.r.l., Sorisole, Italy) with integrated software and the cryoLAB cooling system, equipped with 40 quartz vessels (12-mL capacity) and PTFE covers. Cd was determined using an Agilent 7500cx ICP-MS (Agilent Technologies, Waldbronn, Germany) equipped with an integrated autosampler, a Peltier cooled (at 2° C.) Scott-type quartz spray chamber, a MicroMist nebuliser, a collision cell, and Ni cones. The instrument is controlled and data collected using MassHunter Workstation software, version B.01.01 (Agilent Technologies, 2010). The laboratory facilities, designed for routine measurement of trace elements, operate under positive pressure maintained by a HVAC (heating, ventilating, and air conditioning) system combined with HEPA filters.

Ultrapure water (18 MΩ cm), obtained with a GenPure system (TKA Wasseraufbereitungssysteme GmbH, Niederelbert, Germany), was used for dilution of all solutions. All standard solutions were prepared from a stock standard solution obtained from PlasmaCAL (SCP Science, Quebec, Canada). Analytical grade nitric acid (65%, Merck, Darmstadt, Germany) was used after purification by sub-boiling distillation in SubPUR sub-boiling system (Milestone S.r.l., Sorisole, Italy). All quartz vessels and polypropylene containers were cleaned with detergent solution, soaked in 10% HNO₃ for 24 h, and rinsed with ultrapure water three to five times.

### Acid Digestion of the Samples

0.050 mL of each biological sample was mixed with 0.200 mL of ultrapure 65% nitric acid and 1 mL ultrapure water added in quartz vessel. Microwave digestion was performed in UltraCLAVE IV according to the described procedure (Analytical Protocol AP604-UltraCLAVE, Chapters 4.4-4.9). Due to small amount of sample and reacting acid, the microwave digestion program was modified as shown in Table 1. Following digestion, samples were diluted with ultrapure water to a total volume of 5 mL (i.e. 100 fold dilutions).

TABLE 1
Temperature, time, microwave power and pressure conditions for
digestion of the cell supernatants/lysates in the UltraCLAVE high-pressure
microwave system.

| | T (min:s) | E (W) | T1 (° C.) | T2 (° C.) | P (bar) |
|---|---|---|---|---|---|
| 1 | 3:30 | 700 | 70 | 70 | 100 |
| 2 | 15:00 | 1000 | 180 | 70 | 100 |
| 3 | 10:00 | 1000 | 250 | 70 | 140 |
| 4 | 30:00 | 1000 | 250 | 70 | 140 |
| cooling | 40:00 | 0 | 30 | 70 | 20 |

### Determination of Cd by ICP-MS

Prior to measurements, 2 mL of the acid digested and diluted sample solutions were pipetted into PP vials (V=5 mL; Sarstedt, Germany) and 50 μL of the internal standard solution (ρ=80 μg/L) were added (the final concentration of the internal standards, Ge, Rh, Lu and Tb, in the samples introduced to ICP-MS was 2 μg/L). Prior to measurements (≈60 min), Agilent 7500cx instrument was adjusted to working conditions. Measurements were performed using the instrument's Octopole Reaction System (ORS) and He collision mode (“He-mode”). Sensitivity and stability of the system was checked using the multi-element tune solution (Li, Mg, Co, Y, TI and Ce in 1% HNO₃, ρ=1 μg/L) and formation of oxides (¹⁴⁰Ce¹⁶O⁺/¹⁴⁰Ce⁺=1.2%) and double charged ions (¹⁴⁰Ce²⁺/¹⁴⁰Ce⁺=1.26%) was controlled. Final ICP-MS operating conditions are given in the Table 2. ¹¹¹Cd isotope was measured and ¹⁰³Rh was finally chosen to correct for instrumental drift and matrix effects. Cadmium concentration measurements were also performed in samples treated with medium only (without QDot®655 formulations) for background subtraction purposes.

TABLE 2
Agilent 7500cx operating conditions and data acquisition parameters.

| Parameter | Setting |
|---|---|
| RF Power (W) | 1550 |
| Outer gas flow (L/min) | 15 |
| Sample Depth (mm) | 8.0 |
| Torch-H (mm) | 0.2 |
| Torch-V (mm) | −0.3 |
| Makeup gas flow (L/min) | 0.18 |
| Carrier gas flow (L/min) 0.90 | 0.90 |
| Nebulizer pump (rps) | 0.08 |
| Helium collision gas flow (mL/min) | 3.4 |
| Extract lens 1 voltage (V) | 0.5 |
| Extract lens 2 voltage (V) | −131 |

### Data Analysis

For standard curves, blank values were subtracted from raw data and standard curves were drawn in Microsoft Office Excel 2007 as XY Scatter charts. Linear trendline was applied to the curves, crossing the zero point.

In fluorescence measurements, blank values were subtracted and concentrations (ng/μL) of QDots in samples were calculated from standard curve equation. The results were shown as mean values±SD.

### Results

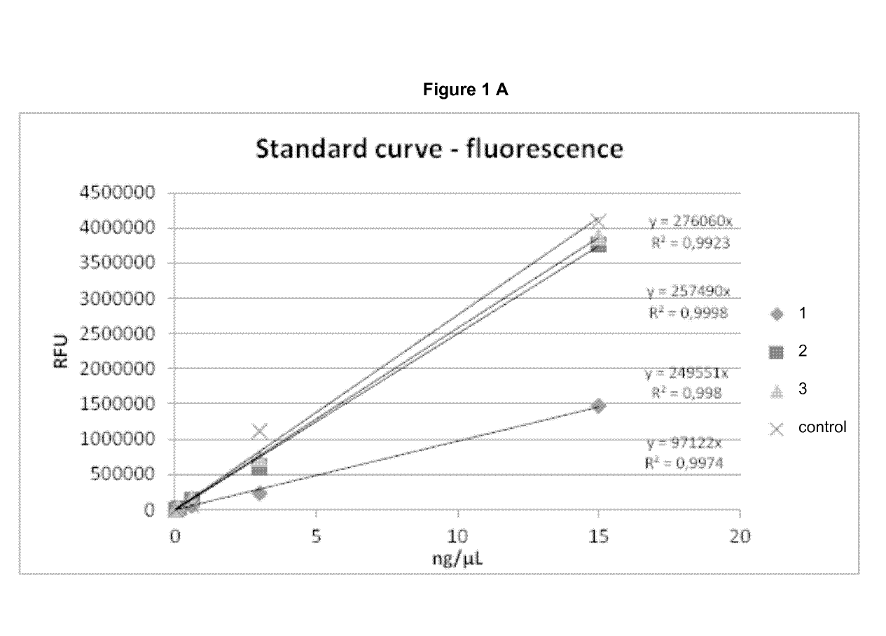

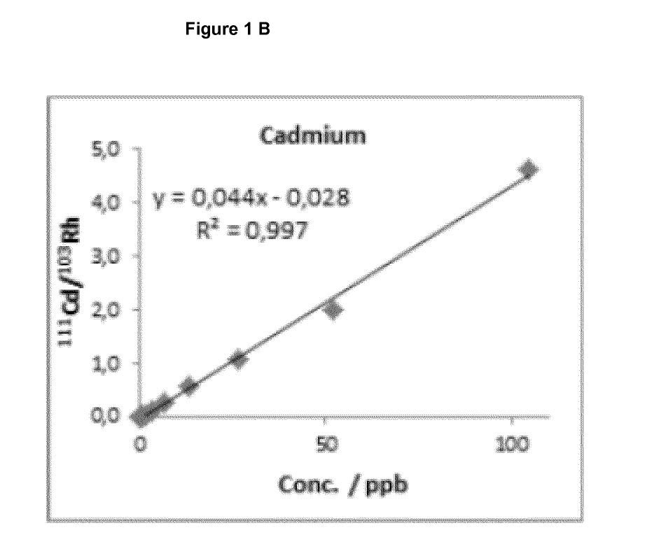

Dispersion of QDot®655 formulations on Caco-2 cell monolayers determined by photography under UV light is shown in FIG. 1. A dose dependent increase in fluorescence was seen on the apical side, while the lower compartment did not show any fluorescence, meaning that no QDot®655 were passing the cell layer. Furthermore, it was evident that pharmaceutical formulation 1 showed a significantly lower fluorescence signal as compared to QDot®655 in borate buffer or formulations without D,L tocopherol. The standard curve for the fluorescence measurement showed linearity between 15 ng/μL and 0.024 ng/μL allowing for comparison of the quantity of QDots (FIG. 1A). There was only a minimal difference in the fluorescence intensity between QDot®655 in borate buffer and pharmaceutical formulations 2 and 3, while the pharmaceutical formulation 1 gave a significantly lower signal as already seen in FIG. 1. No quantifiable levels of QDot®655 at the basal sides of Caco-2 cells were detected following 1 h or 3 h incubation. Therefore, it can be concluded that all tested pharmaceutical formulations have low permeability.

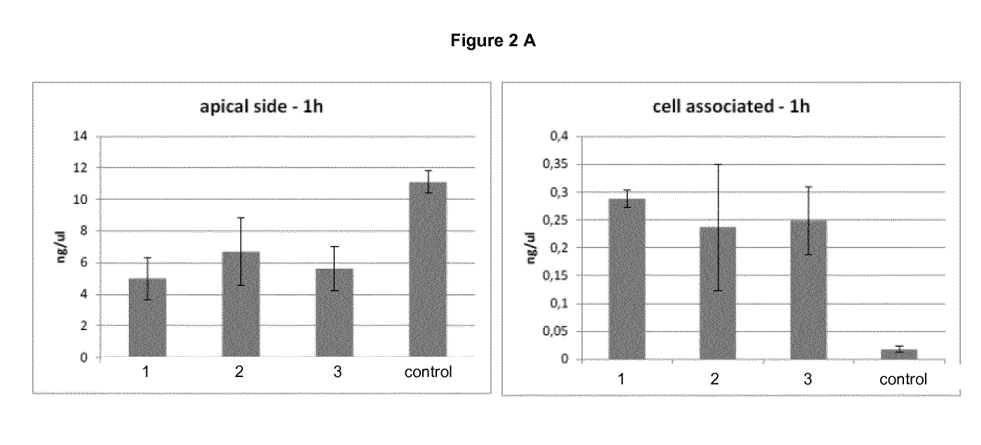

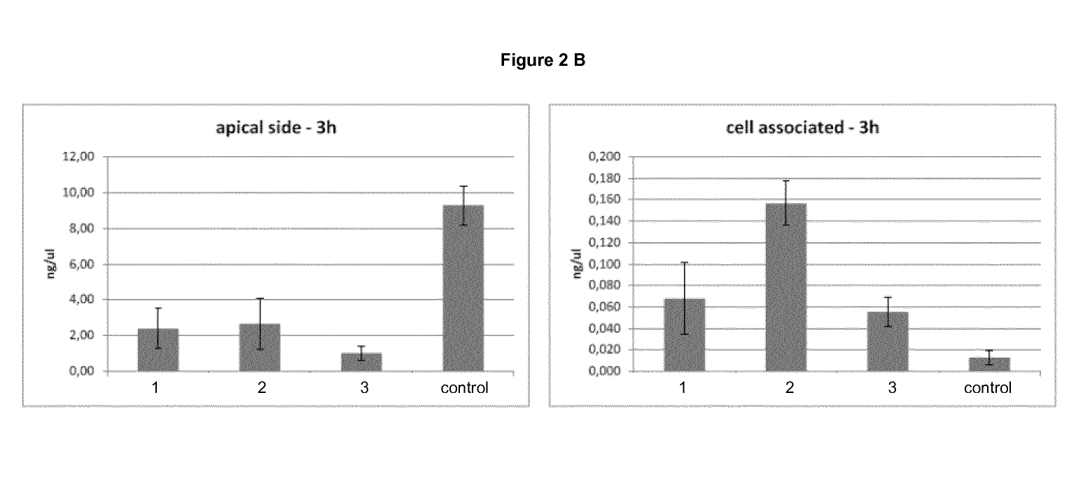

Concentrations of quantum dot formulations at the apical side, as well as cell-associated concentrations determined by fluorimetric measurements, are shown in FIG. 2. At 1 h, concentrations at the apical side did not differ significantly between pharmaceutical formulations, although formulation 1 produced the weakest signal. Cell-associated concentration at the same time point was significantly lower for the sample in borate buffer compared to the three poloxamer containing formulations.

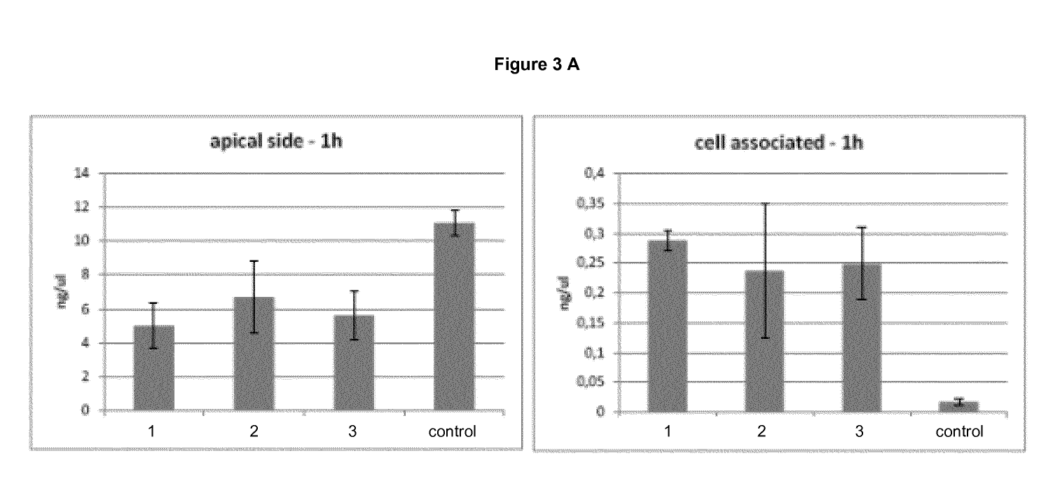

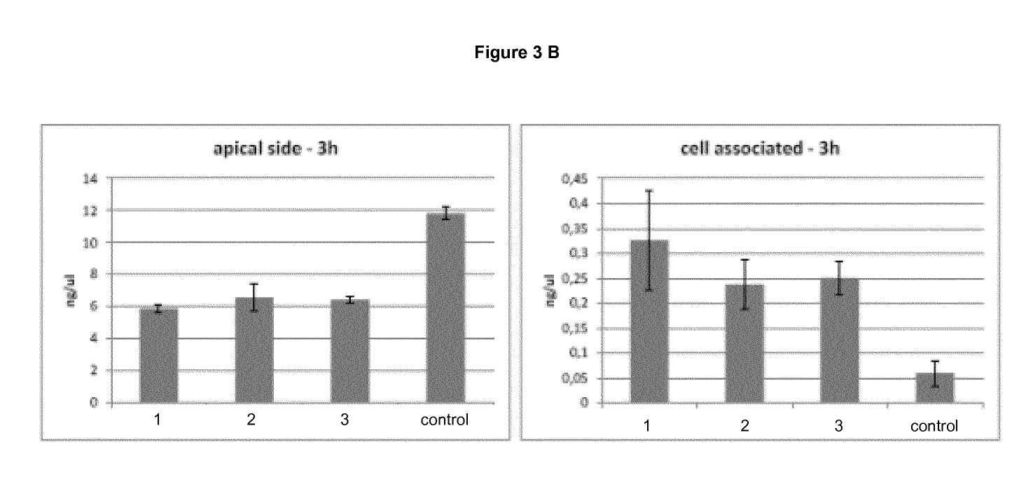

At 3 h, the remaining fluorescence at the apical side was the greatest with the borate buffer containing sample, whereas all poloxamer containing formulations had significantly weaker signals. Cell-associated concentration of pharmaceutical formulation 2 at 3 h was increased compared to 1 h, and higher than for other pharmaceutical formulations at 3 h, indicating most probably greater/more powerful attachment to the cells. Although, 15 ng/μL of each QDot®655 formulation was added to Caco-2 cells, the sums of QDot®655 concentrations at the apical side and cellular level for pharmaceutical formulations 1, 2 and 3 (FIG. 2) were below 15 ng/μL, suggesting that some amount of QDot®655 was washed-out during washing steps. This also indicates that the pharmaceutical formulations did not accumulate inside the cells, but were rather associated with cellular membranes from the outside. The fluorescence signal was clearly associated with the cell layer in the poloxamer containing formulations, while QDot®655 in borate buffer was dispersed in the medium and only slightly attached to the cells (FIG. 2, graphs on the right). The corresponding Cd concentrations in the samples are shown in FIG. 3. The conclusions regarding permeability and cellular association for the above mentioned inventive formulations correspond to the conclusions made on basis of fluorimetric measurements. At 3 h a decrease in fluorescence for poloxamer containing formulations was seen, same as for the Cd level, suggesting a potential instability and degradation of these pharmaceutical formulations due to longer contact with the cells.

### Conclusions

Since there were no quantifiable levels of QDot®655 at the basal side of Caco-2 cells measured in the A2B direction and determined by both methods (fluorimetric measurement and determination of Cd concentration), it can be concluded that all the quantum dot containing formulations have low permeability. In addition, pharmaceutical formulations 1, 2 and 3 tended to be cell associated since, following washing steps, quantifiable levels of QDot®655 and Cd were measured in those cell lysates, compared to borate buffer.

No significant difference following 1 h and 3 h incubation was observed with respect to permeability and cellular association. However, lower fluorescence intensity, with the same Cd concentration, was observed for pharmaceutical formulations 1, 2 and 3 following 3 h in comparison to 1 h incubation. These data suggest a potential instability of QDot®655 in those pharmaceutical formulations due to longer contact with the cells.

### Example 9: Animal Study to Test Local Retention after Injection of an Inventive Formulation

The purpose of this study was to evaluate two pharmaceutical formulations (F1 and F2) according to the invention in comparison to a buffer solution of QDots® 655 (F0) with respect to tissue retention time and possible local effects. Fluorescence at the injection site was determined by photo imaging and fluorescence microscopy over time and histopathological evaluation of inflammatory signs at the local injection site was performed. Obtained data demonstrate that, following intradermal administration, both tested pharmaceutical formulations (F1 and F2) are retained at the injection site longer and send brighter fluorescence signals from the surface side of skin in comparison to F0. There was no significant difference in fluorescence signal intensity and duration between F1 and F2. This study has been performed according to Good Scientific Practice.

### Materials and Methods

### Mice

Male CD1 mice, Charles River (Italy); four weeks old at arrival (BW 24-26 g). Animal related research was conducted in accordance with regional Animal protection legislation (Croatian Official Gazette, NN 135/2006, NN 37/2013 and NN 55/13) and European directive (2010/63/EU) for the protection of animals used for scientific purposes.

The used animal facility is AAALAC (Association for Assessment and Accreditation of Laboratory Animal Care) accredited. Mice are housed in the following ambient conditions: temperature 22° C.±2, with relative humidity 55%±10, about 15-20 air changes per hour and 12 hours artificial lighting and 12 hours darkness per day (7 am lights on-7 pm lights off). Animals have free access to food SDS VRF1 (P), UK and free access to water, distributed in bottles (TECNIPLAST), and filled with drinking water coming from the municipal water main.

All animals were observed daily for changes in behavior, or any signs of distress or toxicity. All injection sites were inspected visually for signs of local intolerance. On the day indicated, animals were euthanatized by administration of an overdose of anaesthetic.

### Preparation of Formulations

Nanoparticles: QDot®655 with Concentration of 4.3 μM=4.3 nmol/ml
Medium: 50 mM borate buffer, pH 8.3
50 μl of injection volume should contain 15 μg of QDots. Following formulations have been prepared:
558 mg of borate buffer (50 mM, pH 8.3) were added to
Batch 22-70: 7441 mg of formulation consisting of 18% Lutrol F127/5% Lutrol F68/70% Aqua ad injectabilia.
Batch 22-71: 7441 mg of formulation 18% Lutrol F127/10% Lutrol F68/65% Aqua ad injectabilia.
   558 mg of Qdots®655 were added to
Batch 22-72: 7441 mg formulation consisting of 18% Lutrol F127/5% Lutrol F68/70% Aqua ad injectabilia
Batch 22-73: 7441 mg of formulation 18% Lutrol F127/10% Lutrol F68/65% Aqua ad injectabilia.
Batch 22-74: 7441 mg of borate buffer as unformulated control solution
   7 ml of each formulation are provided for injection experiments. 1 ml was stored as back-up formulation. Storage of formulated and control preparations take place at 2° C. to 8° C. and under light protected conditions. All formulations are liquid at 2° C. to 8° C., viscous at ambient temperatures and semi-solid at body temperature, respectively.

### Used Formulations

F0=buffer control; quantum dots dissolved in 50 mM borate buffer, 15 μg/50 μL; batch 22-74
F1=Lutrol formulation with QDots15 μg/50 μL; batch 22-72;
Control 1=vehicle control for F1; Lutrol formulation without Dots; batch 22-70;
F2=Lutrol formulation with Qdots15 μg/50 μL, batch 22-73;
Control 2=vehicle control for F2; Lutrol formulation without Qdots; batch 22-71;

### Anesthetics

Ketamine hydrochloride (Narketan, Vetoquinol, Bern, Switzerland).

Xylazinehydrochloride (Rompun, Bayer, Germany).

### Injection Device

1 ml syringes; BD Plastipak; ref 300013, LOT1211009 (exp 10/2017);

Sterile, single use needles; BD MICROLANCE Kanuele, 27 G 3/4 0.4×19 mm and 25 G, Becton Dickinson GmbH, PZN: 3086999.

### UV Lamp and Microscopes

Two identical lamps were used; Vilber Lourmat VL-6.LC at 365 nm.

Light microscope Imaged with AxioCam ICc1 camera (Zeiss).

Confocal microscope (LSM 510 META, Zeiss). Images were acquired using a 458 nm laser for excitation and 585 nm long-pass filter for detection.

Photo camera: Canon, model EOS 350D.

Photo lens: Tamron, EF 28-75 f2.8.

Software for digital photo images editing: Adobe Lightrom v5.

### Experimental Procedures

### Intradermal Injection:

Mice were anesthetized in order to perform intradermal injections. The dorsolateral skin of the animal was carefully shaved with electric clippers to remove the hair. The sites were swabbed with 70% ethanol. The needle was inserted into the skin, bevel up, and held nearly parallel to the plane of the skin without aspiration. A properly performed intradermal injection resulted in a small, round skin welt. Each animal received three intradermal injections of pharmaceutical formulations (formulation, control and F0) on the shaved back. The injection volume of each formulation was 50 μL.

### Subcutaneous Injection:

The dorsolateral skin on the back of the animal was carefully shaved with electric clippers to remove the hair and cleaned with 70% ethanol. The needle was inserted parallel to the skin of the animal, aspirated to ensure proper placement, followed by injection of the pharmaceutical formulations.

Each animal received three subcutaneous injections of formulations (formulation, control and F0). The injection volume of each pharmaceutical formulation was 50 μL.

### Sampling/Photos

At indicated days after application, animals (five per group) were anesthetized i.p. with a combination of ketamine hydrochloride and xylazinehydrochloride. Blood was collected by puncturing the v. jugularis and a. carotis communis into BD tubes with serum separator. Following blood collection, skin from the back area was exposed to UV light and photos taken using a digital SLR camera. Magnification was 0.5×, calculated according to the formula M=f/(g−f); M=magnification, f=the focal length of the lens, g=object distance (between object and lens). For assessment of intradermal application photos were taken both from the surface side and from the subcutaneous side. For assessment of subcutaneous application skin was exposed to UV light from the subcutaneous side and only photos of the subcutaneous side were taken.

Two cryosections were prepared from each skin sample harboring the injection site; skin samples were embedded on the cut edge so that epidermis, dermis and subcutis were revealed. A section encompassing the entire skin thickness was mounted onto a slide and acetone fixed. One of the cryosections was immediately analyzed by confocal microscopy for the presence of fluorescence signal. The other section was stained with hemalaun-eosin and analyzed by light microscopy for signs of inflammation at the injection site. The remaining tissue was placed into 10% formalin.

### Data Analysis and Statistical Evaluation

Histological analysis was performed as follows:

Digital pictures were taken from each skin sample analysed by confocal microscopy. Pictures were taken at 100× magnification. The intensity of the fluorescence signal was described using a semi-quantitative descriptive method: no fluorescence (0), weak fluorescence (1), moderate fluorescence (2), and bright fluorescence (3).

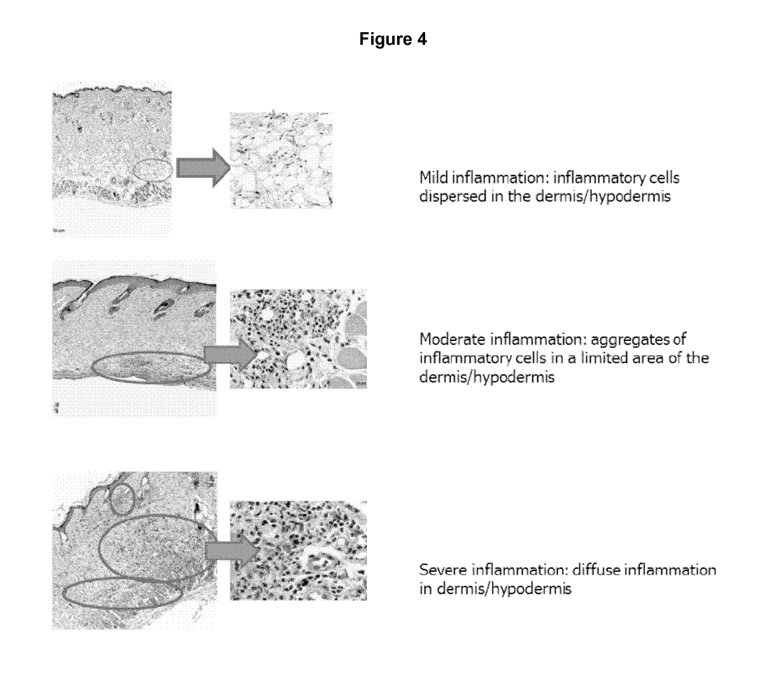

Inflammation was defined as an accumulation of neutrophils and mononuclears in the dermis and/or hypodermis. The site of inflammation was described as “inflammation in dermis” and “inflammation in hypodermis”. The intensity of the inflammatory reaction was graded as none (0), mild (1), moderate (2) and severe (3) (FIG. 4). Dispersed inflammatory cells in the dermis/hypodermis were regarded as “mild inflammation”. Formation of aggregates of inflammatory cells in a limited area of the dermis/hypodermis was regarded as “moderate inflammation”. Presence of diffuse neutrophils and mononuclears in the dermis/hypodermis was regarded as “severe inflammation”. Intermediate scores (0/5, 1/2 and 2/3) were used in some cases. The median for each group was calculated.

### Results

### Intradermal Injection

### Fluorescence of QDots® 655

The fluorescence signal was evaluated from the cutaneous and the subcutaneous side according to the semi-quantitative descriptive scoring system (0=no fluorescence; 1=weak fluorescence; 2=moderate fluorescence; 3=bright fluorescence) over a period of five days (cutaneous side) and seven days (subcutaneous side) following intradermal application. Two observers in parallel have scored the fluorescence signal. Table 3 summarizes the fluorescence intensity evaluated from the cutaneous side while table 4 summarizes the corresponding scores from the subcutaneous side.

As seen from Table 3, when evaluated from the cutaneous side, pharmaceutical formulations F1 and F2 have shown a prolonged signal, while fluorescence of QDots®655 dissolved in buffer (F0) faded within one day. Contrary to that, as seen from Table 4, subcutaneous evaluation indicated a prolonged signal of QDots® 655 dissolved in buffer (F0) when compared to pharmaceutical formulations F1 and F2. These data indicate that QDots® 655 dissolved in buffer did not remain at the site of the injection, but have penetrated the subcutaneous space following intradermal injection, while pharmaceutical formulations F1 and F2 maintained the fluorescence signal at the local injection site.

TABLE 3
Evaluation of fluorescence from cutaneous side
following intradermal injection of QDots ®655.
Scoring scheme: 0 = no fluorescence; 1 = weak fluorescence;
2 = moderate fluorescence; 3 = bright fluorescence.

| Time | | | | | | | |
|---|---|---|---|---|---|---|---|
| post | | Formulations/number of animals | | | | | |
| injection | score | F0 | F1 | CTRL1 | F0 | F2 | CTRL2 |
| 24 h | 0 | | | 5 | | | 5 |
| | 1 | | | | | | |
| | 2 | | 1 | | | | |
| | 3 | 5 | 4 | | 5 | 5 | |
| Comments | | — | — | — | — | — | — |
| 72 h | 0 | 2 | 1 | 5 | | 1 | 5 |
| | 1 | | | | 3 | 1 | |
| | 2 | 3 | 1 | | 2 | 2 | |
| | 3 | | 3 | | | 1 | |
| Brownish tissue | | — | 1/5 | 1/5 | — | 1/5 | — |
| coloration at | | | | | | | |
| administration site | | | | | | | |
| Day 5 | 0 | 4 | 4 | 5 | 3 | 3 | 5 |
| | 1 | 1 | 1 | | 2 | | |
| | 2 | | | | | 1 | |
| | 3 | | | | | 1 | |

| | | | | | | |
|---|---|---|---|---|---|---|
| Brownish tissue | 2/5 | 2/5 | — | 3/5 | 2/5 | — |
| coloration at | | | | | | |
| administration site | | | | | | |

TABLE 4
Evaluation of fluorescence from subcutaneous side
following intradermal injection of QDots ®655.
Scoring scheme: 0 = no fluorescence; 1 = weak fluorescence;
2 = moderate fluorescence; 3 = bright fluorescence.

| Time | | | | | | | |
|---|---|---|---|---|---|---|---|
| post | | Formulations/number of animals | | | | | |
| injection | score | F0 | F1 | CTRL1 | F0 | F2 | CTRL2 |
| 24 h | 0 | | 1 | 5 | | 3 | 5 |
| | 1 | | 4 | | 1 | 2 | |
| | 2 | 3 | | | 1 | | |
| | 3 | 2 | | | 3 | | |
| Comments | | — | — | — | — | — | |
| 72 h | 0 | 1 | | 5 | 2 | 5 | 5 |
| | 1 | | | | 3 | | |
| | 2 | 1 | | | | | |
| | 3 | 3 | | | | | |
| Brownish tissue | | — | 5/5 | — | — | 3/5 | — |
| coloration at | | | | | | | |
| administration site | | | | | | | |
| Day 5 | 0 | 5 | 5 | 5 | 4 | 5 | 5 |
| | 1 | | | | 1 | | |
| | 2 | | | | | | |
| | 3 | | | | | | |
| Brownish tissue | | 4/5 | 4/5 | — | 4/5 | 4/5 | — |
| coloration at | | | | | | | |
| administration site | | | | | | | |
| Day 7 | 0 | | 5 | 5 | 3 | 4 | 5 |
| | 1 | 5 | | | 2 | 1 | |
| | 2 | | | | | | |
| | 3 | | | | | | |

| | | | | | | |
|---|---|---|---|---|---|---|
| Comments | — | — | — | — | — | — |

### Confocal Microscopy

In order to further quantify the fluorescence signal and evaluate the tissue distribution of QDots® 655, tissue samples of intradermal injection sites were analysed by confocal microscopy.

Table 5 and Table 6 summarize the results of fluorescence signal evaluation based on photographs taken with confocal microscope. High variation of fluorescence signal was obtained. While a bright signal was seen in all groups 24 h following injection, variable fading of the fluorescence intensity was seen in the pharmaceutical formulations F1 and F2 (at 72 h, 5 days and 7 days), as well as within the buffer formulation F0 (at 72 h, 5 days and 7 days). Both vehicle controls (CTRL1 and CTRL2) did not show a fluorescence signal in any of the groups (data not shown).

TABLE 5
Fluorescence intensity as determined by confocal microscopy following
intradermal injection.

| Time | | |
|---|---|---|
| post | | |
| injec- | | |
| tion | F0 and F1 | F0 and F2 |
| 24 h | Bright signal for F0 and F1 | Bright signal for F0 and F2 |
| 72 h | Moderate to bright signal for F0 | Moderate to bright signal for F0 |
| | Weak to moderate signal for F1 | Weak signal with F2 |
| Day | Moderate to bright signal for F0 | Moderate to bright signal for F0 |
| 5 | Weak to moderate signal with | Variable signal for F2 (without |
| | F1 | signal or weak; one animal with |
| | | bright signal) |
| Day | Moderate to bright signal for F0; | Variable signal for F0; animals |
| 7 | animals without signal (1/5) | without signal (3/5) |
| | Weak signal or without signal | Weak signal or without signal |
| | (4/5) with F1 | (3/5) with F2 |

TABLE 6
Fluorescence intensity following intradermal injection, median values,
n = 5.

| Time post | F0 | F1 | F2 | F3 |
|---|---|---|---|---|
| injection | (median) | (median) | (median) | (median) |
| Day 1 | 3 | 3 | 3 | 3 |
| Day 3 | 2 | 1.5 | 2 | 1 |
| Day 5 | 2 | 1 | 2 | 1 |
| Day 7 | 3 | 0 | 0.5 | 0 |

### Evaluation of Inflammation

Acute inflammation, defined by the presence of neutrophils among other cell types, was found in all skin samples containing QDots®655. Intradermal application of F0 and both F1 and F2 formulations, induced a mild acute inflammatory reaction at 24 h, characterized by the presence of single inflammatory cells and small accumulations of inflammatory cells in the dermis. Inflammation spread throughout the dermis and into the hypodermis during the observation period. Macrophages, some of which were pigmented, could be identified among inflammatory cells, at 72 h post F1/F2 application until the end of the observation period. The intensity of inflammation varied among animals in the group. Generally, F2 induced a milder inflammation in comparison to F0 and F1. The control induced a mild inflammation that resulted in scar formation along the needle track at the end of the observation period. Table 7 summarizes inflammatory scores following intradermal injection of QDots® 655 formulations F1, F2, as well as buffer formulation (F0).

TABLE 7
Inflammation intensity following intradermal
injection, median values, n = 5.

| Time post | F0 | Controls | F1 | F2 |
|---|---|---|---|---|
| injection | (median) | (median) | (median) | (median) |
| 24 h | 1 | 1 | 1 | 1 |
| 72 h | 2 | 1 | 2 | 1.5 |
| Day 5 | 2 | 1 | 2.5 | 1 |
| Day 7 | 2 | 1 | 1 | 1 |

### Subcutaneous Injection

### Fluorescence of QDots® 655

The fluorescence signal was evaluated from the subcutaneous side according to the semiquantitative descriptive scoring system (0=no fluorescence; 1=weak fluorescence; 2=moderate fluorescence; 3=bright fluorescence) over a period of seven days following subcutaneous application. No signal was detectable from the cutaneous side. Brownish coloration of the tissue was observed in two animals from subcutaneous side seven days following application of F2. Table 8 summarizes the fluorescence intensity evaluated from the subcutaneous side. Pharmaceutical formulations F1 and F2 gave a slightly weaker signal when compared to the buffer formulation (F0). Individual photographs show that the buffer formulation gave a more dispersed signal, while F1 and F2 formulations appear to be more localized around the injection site.

TABLE 8
Evaluation of fluorescence from subcutaneous side following
subcutaneous injection of QDots ®655. Scoring scheme:
0 = no fluorescence; 1 = weak fluorescence; 2 = moderate
fluorescence; 3 = bright fluorescence.

| Time | | | | | | | |
|---|---|---|---|---|---|---|---|
| post | | Formulations/number of animals | | | | | |
| injection | score | F0 | F1 | CTRL1 | F0 | F2 | CTRL2 |
| 24 h | 0 | | | 5 | | | 5 |
| | 1 | | 1 | | | 1 | |
| | 2 | 2 | 4 | | 1 | 2 | |
| | 3 | 3 | | | 4 | 2 | |
| Comments | | — | — | — | — | — | — |
| Day 3 | 0 | | 2 | 5 | | 4 | 4 |
| | 1 | 1 | 3 | | 2 | | |
| | 2 | 4 | | | 2 | | |
| | 3 | | | | | | |
| Comments | | — | — | — | — | — | — |
| Day 7 | 0 | 5 | 3 | 5 | | 3 | 5 |
| | 1 | | 2 | | 3 | 2 | |
| | 2 | | | | 2 | | |
| | 3 | | | | | | |

| | | | | | | |
|---|---|---|---|---|---|---|
| Brownish tissue | — | — | — | — | 2/5 | — |
| coloration at | | | | | | |
| administration site | | | | | | |

### Confocal Microscopy

The analysis of samples by confocal microscopy confirmed the macroscopic findings. While at 24 h following injection, a bright signal could be seen in all three pharmaceutical formulations (F0, F1, F2), it was observed that the signal faded away over the period of seven days. Both vehicle controls (CTRL1 and CTRL2) did not show a fluorescence signal in any of the groups.

TABLE 9
Fluorescence intensity determined by confocal
microscopy following subcutaneous injection.

| Time | | |
|---|---|---|
| post | | |
| injection | F0 and F1 | F0 and F2 |
| 24 h | Bright signal for F0 and F1, | Bright signal for F0 and F2 |
| | some animals with weaker | |
| | signal | |
| 72 h | Moderate signal for F0 | Moderate to bright signal for F0 |
| | Fading of signal with F1 | Fading of signal with F2 and |
| | and some animals without | some animals without signal * |
| | signal | |
| Day 7 | No signal for F0 and F1 | Weak or moderate signal for F0; |
| | | some animals without signal |
| | | Weak signal for F2 and some |
| | | animals without signal |

* Group of 4 animals, since one animal from the group was found dead.

TABLE 10
Fluorescence intensity following subcutaneous
injection, median values, n = 5.

| Time post | F0 | F1 | F0 | F2 |
|---|---|---|---|---|
| injection | (median) | (median) | (median) | (median) |
| Day 1 | 3 | 1 | 2 | 3 |
| Day 3 | 2 | 2 | 1.5 | 0* |
| Day 7 | 0 | 0 | 1.5 | 0.5 |

*Median is given for the group of 4 animals, since one animal from the group was found dead.

### Evaluation of Inflammation

Subcutaneous application of F0, and both F1 and F2 formulation, induced an acute inflammatory reaction in the hypodermis already on day one post application. The inflammatory reaction was more pronounced in animals treated with F1 and F2 in comparison to F0. Occasionally, small accumulations of brownish amorphous material were identified in the hypodermis/subcutis. Macrophages, some of which were pigmented, were observed at three days post injection. The intensity of the inflammatory reaction decreased over time in all groups, but neutrophils could be identified throughout the entire duration of the experiment. However, individual variations were observed among animals. The control induced a mild reaction that was resolved by the end of the observation period.

TABLE 11
Inflammation intensity following the subcutaneous
application, median values, n = 5.

| Time post | F0 | F1 | F0 | F2 |
|---|---|---|---|---|
| injection | (median) | (median) | (median) | (median) |
| 24 h | 1 | 1 | 2.5 | 2 |
| 72 h | 0 | 1 | 0 | 1* |
| Day 7 | 0 | 0 | 1 | 1 |

*Median is given for the group of 4 animals.

### Clinical Observations

Animal appearance, as well as food and water intake, was normal in all study groups. The animals displayed normal species specific behaviour. There were no macroscopic signs of inflammation, or necrosis at injection sites. Brownish tissue coloration at administration site after intradermal injections was observed as small spots from cutaneous side, as stated in Table 2 with results.

Note: *one animal has been found dead and excluded from further evaluation due to cannibalism.

### Discussion and Conclusions

Single intradermal or subcutaneous application of QDots® 655 dissolved in buffer (F0) and two different QDots® 655 formulations marked as F1 and F2 resulted in visible red fluorescence signal after exposure to UV light. Close-up photographs of skin surface (cutaneous) side following intradermal injection, have clearly shown that the signal associated with the F0 formulation was less intensive, or mostly not visible, at 72 h hours post application, whereas both pharmaceutical formulations F1 and F2 were still clearly visible at 72 h post application in the majority of the animals. Moreover, upon exposure to UV light five days post application, fluorescent spots of the F2 formulation were still visible in two animals. No fluorescent signals were present seven days post application. However, inspection of the subcutaneous side, following intradermal injection, revealed intensive fluorescence signals 24 h post application both for F0 and F1/F2 formulations. At 72 h the signal on the subcutaneous side was brighter for F0 than for F1 and F2. Fluorescence signals were practically not present at days three to seven post application. Therefore, it can be concluded that QDots® 655 dissolved in buffer penetrate rapidly through the dermis into the subcutaneous space, while the pharmaceutical formulations F1/F2 withhold the QDots® 655 at the site of the (intradermal) injection. Confocal microscopy confirmed the macroscopic results: at 24 h following intradermal injection of F0, a bright florescence signal was observed, that progressively faded away in the course of seven days post application. The F1 formulation gave a bright signal at 24 h, which faded away over time, but a weak signal was still present at seven days post application. The F2 formulation also gave a bright signal at 24 h post application, but faded away during the observation time. Quantum Dots were distributed non-homogeneously mostly throughout the epidermis when formulated, and no red fluorescence was observed in hair follicles. Quantum Dots were found in the dermis, and during the observation period they were progressively diffusing towards the hypodermis. F0 and F1/F2 induced an acute inflammatory reaction characterized by accumulation of inflammatory cells at 24 h post application. Intradermal injection of F0 and F1 caused a brighter inflammation than F2. Injection of pharmaceutical formulations corresponding to F1 and F2, but without QDots® 655, induced a mild inflammation resulting in scar formation along the needle track by the end of observation period.

Overall, it can be concluded that pharmaceutical formulations F1/F2 of Quantum Dots maintain a prolonged fluorescence signal at the site of injection following intradermal injection, accompanied by a mild inflammatory reaction, that fades away in the course of the observation period.

Following single subcutaneous injection, upon exposure to UV light, the fluorescence signal could be seen at 24 h post application with F0 (buffer) and with both pharmaceutical formulations on closeup photographs. At 72 h the fluorescence signal on the subcutaneous side was brighter in the F0 group than in F1/F2 groups. Fluorescence signals were no longer visible at day seven after application. Also, brownish coloration of tissue was observed at some injections sites five days post injection. Confocal microscopy showed that, following subcutaneous injection of F0, a bright fluorescence signal was captured at 24 h post application and progressively faded away in the course of seven days. The F1 formulation also gave a bright signal at 24 h after application, but faded away over time and completely disappeared at day seven.

Similarly, the F2 formulation gave a bright signal at 24 h, getting weaker at 72 h after application, but was still present but very weak seven days post application. Fluorescence signals were diffusely found in the hypodermis/subcutis. Contrary to intradermal injection, higher individual variations were detected among animals from the same group following subcutaneous injection. This is probably due to dispersion of the applied volume, since the subcutaneous space in mice is a large virtual space. As visible in close-up photos, QDots® 655 tend to spread throughout the subcutis due to high mobility of skin in mice.

Generally, the fluorescence signal was obviously more dispersed for F0 than for F1/F2. Additionally, mainly at later time points, it was not possible to detect with accuracy the exact injection site in some skin samples, which made the sampling for confocal microscopy more difficult. Nevertheless, the signal intensity recorded at close-up, was mostly in concordance with confocal microscopy. Subcutaneous application of F0 and F1/F2 induced an acute, mild to moderate inflammatory reaction in the deep hypodermis/subcutis already during the first 24 hours post application. Intensity of the inflammatory reaction decreased in all groups following subcutaneous application already at 72 hours after application, although there were individual variations among animals. Control formulations, corresponding to F1 and F2, but without Quantum Dots, also induced a mild inflammation that was resolved by the end of the observation period. It should be taken into account that mild inflammation is always present due to needle insertion into the skin.

Several Conclusions can be Made from the Study:
1. Following intradermal application, F0 is diffusing to the subcutaneous side much easier than F1/F2. This explains a brighter fluorescence signal when inspected from the subcutaneous side. Close-up photos showed that, following intradermal injection, F1/F2 formulations had a lower penetration rate towards the subcutaneous side in comparison to F0.
2. There was no significant difference in fluorescence signal intensity and duration between F1 and F2 on the cutaneous and subcutaneous side. The mouse skin consists of an external epithelium (epidermis), a thick layer of connective tissue (dermis), and a layer of adipose tissue (hypodermis or panniculus adiposus). A thin layer of striated muscle separates the skin from other structures. The injection is made into the outer layers of the skin and F1/F2 obviously spread more slowly throughout the dermis during the observation period. Also, there is a higher possibility of some leakage after intradermal application of F1/F2 due to the viscosity of the pharmaceutical formulations, which may also produce some variability. Diffusion of F1 and F2 from the epidermis into deeper dermis/hypodermis was probably limited by the basement membrane and dermal connective tissue, so only brownish coloration of the subcutis was visible at later time points, mostly without fluorescent signal from the subcutaneous side. This was confirmed by observations from the surface side, since fluorescence spots of F1 and particularly F2 were intensive for a longer period of time than F0.
3. Successful injections were easily visible from the surface side, and compared to F0, fluorescent spots from F1 and F2 were well defined, better assembled in more limited areas and more intensive for a longer period of time. Obtained data demonstrate that following intradermal injection, F1 and F2 are better retained at the site of injection and produce a brighter fluorescence signal than F0.
4. Following subcutaneous injection of F0 and F1/F2, the non-homogeneously distributed red fluorescence signal was concentrated in the hypodermis and between the underlying muscle fibers. With time, QDots diffused in both directions—towards the dermis and the subcutis. Anatomical conditions edict a more localized signal following intradermal application, when compared to subcutaneous. However, even after subcutaneous application, F1 and F2 related signal was more homogenous than that of F0. In general, the signal from F0 was longer visible after subcutaneous application than after intradermal application but more dispersed.
5. Generally, mild to moderate inflammatory signs were seen in all injections, even in pharmaceutical formulations without QDots®655.Inflammatory reaction was most prominent at 24 h and stronger following intradermal vs. subcutaneous injection. F2 formulation induced a milder inflammatory reaction than F1. Inflammatory signs faded away in all pharmaceutical formulations with time.

### Example 10: Evaluation of QDot 655 Binding on Human Colon Tissue

Membrane binding of QDot®655 was evaluated in cryo tissue sections of human colon cancer and non-malignant colon epithelium using standard immunohistochemical methods for incubation, washing and detection of QDot®655. For control purposes the membrane marker (CDH17) was used to demonstrate specific membrane binding pattern for the conditions and tissue samples used.

Binding of QDot®655 was probed by using three 0.015% per weight QDot®655 preparations, buffer (PBS) and two poloxamer gel formulations, F4 and F5, respectively. Furthermore the membrane binding experiments of CDH17 and QDot 655 were completed by respective negative controls (antibody isotype control, PBS, Gel-F4 and Gel-F5). Binding studies were executed at room temperature and 37° C.

### A) Manufacturing Protocol of Tested QDot 655 Gel Formulation F4 and F5 1.

### List of Ingredients and Equipment Used:

QDot®655 colloidal solution in 50 mM borate buffer from LTC Life Technologies Corporation, Carlsbad, Calif., US C47013

Kolliphor® P407 BTC Europe GmbH, Burgbernheim

Kolliphor® P188 BTC Europe GmbH, Burgbernheim

Aqua ad injectabilia Brau, Melsungen

### Antibodies:

Human Cadherin-17 MAb (CHD17), mouse monoclonal, clone 141713 (Biomedica (R&D); RD-MAB1919)
Isotype control, mouse IgG1 (DAKO; X0931)

TABLE 12
Composition of final (3.5%) QDot containing formulation F4

| | | net weight |
|---|---|---|
| Excipients and substance | Concentration [%] | in 20 g |
| Kolliphor ® P407 | 18.00% w/w | 3.60 g |
| Kolliphor ® P188 | 5.00% w/w | 1.00 g |
| Aqua ad inj. | 73.50% w/w | 14.70 g |
| purified QDots ®655 | 3.50% w/w | 0.70 g |

The final concentration of quantum dots in the formulation F4 was 0.015% per weight.

TABLE 13
Composition of final (3.5%) QDot containing formulation F5

| | | net weight |
|---|---|---|
| Excipients and substance | Concentration [%] | in 20 g |
| Kolliphor ® P407 | 18.00% w/w | 3.60 g |
| Kolliphor ® P188 | 10.00% w/w | 2.00 g |
| Aqua ad inj. | 68.50% w/w | 13.70 g |
| purified QDots ®655 | 3.50% w/w | 0.70 g |

The final concentration of quantum dots in the formulation F5 was 0.015% per weight.

The final concentration was calculated based on the measured amount of Cd in the initial QDot solution and in the final formulation (no free Cd was found in these solutions). The Cd amount measured in the final formulation was reduced by factor 30 compared to the initial solution. The initial QDot®655 colloidal solution in 50 mM borate buffer of LTC Life Technologies Corporation contained 4.2 μM QDots having a molecular weight of 1000. This information allows calculating the % per weight of nanoparticle in the formulation F4 and F5.

### 2 Procedure: Purification of QDot®655 Original Solution by Repeated Filtration Steps

1 g of QDot®655 original solution was transferred into Vivaspin cartridge and 4 g of Aqua ad injectabilia were added. This Vivaspin cartridge was centrifuged at 3000 g for 30 min at 21° C. Subsequently the Filtrate 1 was removed from the lower part of the cartridge.

Then 4 ml of aqua ad injectabilia were added to the QDot®655 concentrate in the upper compartment of the cartridge, carefully resuspended and again centrifuged (3000 g/30 min/21° C.). Filtrate 2 was removed from the lower part of the cartridge. This procedure was repeated once.

Finally Aqua ad injectabilia was exactly added to the QDot®655 concentrate in the upper compartment of the cartridge (taking the tare weight into consideration) to obtain the initial weight of 1 g. The QDot®655 concentrate was carefully resuspended and stored at 4° C. under light protection in glass vials.

### 3. Preparation Procedure

Kolliphor P407 and Kolliphor P188 were accurately weighted into a glass beaker which was placed in a preheated (70° C.-80° C.) water bath.

After the Poloxamer was completely melted; the appearance was colorless, transparent, homogeneous, and liquid. Aqua ad injectabilia was added to the molten mass and dispersed by stirring. Immediately after dispersing preparation was stored under refrigerated conditions (2° C.-8° C.) until appearance is colorless, homogeneous and transparent. Aqua ad injectabilia was added to obtain final net weight of 50 g of the F4 or F5 carrier gel.

19.3 g of the F4 or respectively F5 carrier gel was accurately weight in a glass beaker and 700 mg of purified QDot®655 suspension was added and slowly mixed until the formulation appears homogeneous, orange red and transparent. The product was transferred into glass vials, sealed and stored under protection from light at a cool place.

For subsequent use the QDot®655 dipeptide quantum dots (100%) were diluted with PBS to yield a final working concentration of 3.5%. QDot 655 gel formulation F5 (7%) were diluted with Poloxamer gel formulation F5 to yield a final working concentration of 3.5%. Prepared dilutions were mixed for 5 min by overhead mixing at room temperature, final dilutions were stored at 4° under light protection.

### B. Study Design and Goal

Aim of this project is the evaluation of the potential degree of membrane binding properties of QDot®655 in an inventive formulation at room temperature (RT, 24° C.) as well as at 37° C. to colon cancer tissue and to non-malignant colon tissue. Therefore the binding of QDot 655 is evaluated for an aqueous buffered QDot 655 solution (QDot®655-buffer) as well as for inventive QDot®655 preparation (QDot 655-gel F4 and QDot®655-gel F5). The analysis allows the comparison of potential membrane binding properties of QDot®655 under various conditions (i.e. buffer, gel, RT, 37° C.) and tissue types (i.e. cancer, non-malignant). This study was planned, performed, monitored, recorded, archived and reported according to the Quality Management System of ORIDIS Biomarkers.

### C. Materials and Methods

### Human Cryo Tissue Samples

Anonymized cryo tissue samples and pathological data of adenocarcinoma of the colon (n=3; table 14) or normal colon epithelia (n=3; resection margins of colon cancer patients) were purchased from the EN/ISO 9001:2008 certified Biobank Graz (Graz, Austria). Tissue histology (table 14) was re-evaluated and confirmed by a trained pathologist. Tumor and non-malignant epithelia tissue area was quantified using Aperio ScanScope software (Leica Biosystems, Germany) in order to quantify the approximate tissue content available for evaluation.

TABLE 14
Colon tissue samples and pathological data.

| | | | Qualified | | | | |
|---|---|---|---|---|---|---|---|
| | | | tissue | | | | |
| Sample | | | size | | | | |
| ID | Histology | Scan file | (mm²) | Age | Gender | Stage | Grade |
| 40935 | Non-malignant | 2571407_3 | 17.8 | 62 | ♂ | — | |
| | epithelia | | | | | | |
| 4792 | Non-malignant | 2571407_5 | 11.3 | 80 | ♂ | — | |
| | epithelia | | | | | | |
| 5152 | Non-malignant | 2571407_8 | 13.6 | 83 | ♀ | — | |
| | epithelia | | | | | | |
| 7545 | adenocarcinom | 2571407_2 | 9.08 | 52 | ♂ | III | G3 |
| 4738 | adenocarcinom | 2571407_4 | 12.8 | 65 | ♀ | III | G§ |
| 26 | adenocarcinom | 2571407_10 | 19.6 | 69 | ♂ | IV | G3 |

### QDot®655 and Antibody Binding

Cryo tissue sections with a thickness of 5 μm have been used for subsequent analysis of QDot®655 binding as well as analysis of CDH17 and the control antibody immune reactivity. Cryo tissue sections were fixed for 10 min in acetone (4° C.), incubated with Bovine Serum Albumin (20 min, 2% (w/v) BSA, RT). Next tissue sections were incubated with QDot 655-buffer (3.5%), QDot®655-gel F4/F5 (3.5%) and controls, respectively for one hour at RT or 37° C. After incubation of the tissue slides with QDot®655 preparation and controls, the tissue slides were washed 3 times for 10 min. Finally slides had been DAPI stained and covered.

Alternatively, after were blocking the endogenous peroxidase activity (20 min, 1 H₂O₂, room temperature) and incubation with Bovine Serum Albumin (20 min, 2% (w/v) BSA, RT) the fixed sections were incubated with CDH17 and control antibody dilution (1 μg/ml, 1 h), respectively. Next, slides were washed (3 times, 10 min) and subsequently incubated with ChemMate Envision HRP mouse/rabbit (bottle A, 30 min) and further processed according to the manufactures guideline. Finally sections were covered with Entellan®.

### Sample Evaluation and Documentation

In the region of interest (adenocarcinoma of the colon or non-malignant colon epithelia), membrane binding of QDot®655-buffer or QDot 655-gel F4/F5 in epithelial cells were evaluated. Digital images of representative areas were captured at a Zeiss Axioplan microscope using an Axiocam MRm camera from Zeiss (Jena, Germany) at a total microscope magnification of 200×.
Combined images of DAPI (Excitation: 365 nm; Emission: 445/50 nm) and QDot®655 fluorescence (Excitation: 546/12 nm; Emission 590 nm) were recorded at a 200× microscope magnification and subsequently analyzed for percentage of luminal membrane showing QDot®655 fluorescence.
Lengths of total luminal membrane and luminal membranes with signal were calculated using image analysis software, ImageScope (Aperio, UK).
Luminal membrane positive for QDot®655 fluorescence were expressed as % of total luminal membrane.
The average membrane binding and standard deviation was calculated for each experimental condition tested (i.e. tissue sample ID, QDot type/control and temperature) based on the three batches analyzed for each experimental condition.
The average membrane binding and standard deviation was calculate for each group of experiments (i.e. tissue type, QDot type/control and temperature)
For statistical comparison of membrane binding between tissue types (i.e. non-malignant colon vs cancer epithelium), temperatures and formulations, membrane binding was compared using Mann-Whitney U test.
Statistical calculations were performed using SigmaPlot® (Systat Software, Germany)

### Results

TABLE 15
Summary of luminal reactivity (%) in non-malignant and cancer
epithelium at room temperature and 37° C. Given are averages
and standard deviations oft he % of luminal membrane reactivity
for three tissue samples and three independent experiments
for each tissue sample for each given condition.

| | RT/24° C. | | 37° C. | |
|---|---|---|---|---|
| Condition | non-malignant | tumor | non-malignant | tumor |
| CDH17 | 95 ± 0 | 95 ± 0 | n.d. | n.d. |
| Isotype control | 0 ± 0 | 0 ± 0 | n.d. | n.d. |
| QDot 655-buffer | 0 ± 0 | 28 ± 20 | 0 ± 0 | 16 ± 26 |
| PBS | 0 ± 0 | 0 ± 0 | 0 ± 0 | 0 ± 0 |
| QDot 655-gel F4 | 0 ± 0 | 22 ± 29 | 0 ± 0 | 67 ± 20 |
| Gel-F4 | 0 ± 0 | 0 ± 0 | 0 ± 0 | 0 ± 0 |
| QDot 655-gel F5 | 0 ± 0 | 13 ± 16 | 0 ± 0 | 26 ± 39 |
| Gel-F5 | 0 ± 0 | 0 ± 0 | 0 ± 0 | 0 ± 0 |

Controls:
None of the negative controls (antibody isotype control, PBS, Gel-F4 and Gel-F5) indicated for any detectable membrane signal, excluding also potential autofluorescence.
CHD17:
Specific membrane immune reactivity to CDH17 was observed in cells of nonmalignant epithelia colon tissue and adenocarcinoma of the colon. The observed membrane reactivity was present in at least 95% of the entire membrane. The negative control (antibody isotype) lacked any detectable membrane reactivity.
QDot 655-buffer solution:
No membrane binding of QDot 655 in PBS (QDot 655-buffer) was observed in non-malignant colon tissue after incubation for 1 hour at RT or 37° C.
A luminal membrane binding of QDot 655-buffer was detected in all three adenocarcinoma samples at RT (QDot 655 binding to 8%-46% of luminal membrane) and in two of three samples (ID 4738 and ID 26) after incubation at 37° C. (QDot 655 binding to 10%-39% of luminal membrane)
Intracellular binding (cytoplasm and nucleus) was observed in non-malignant colon tissue and adenocarcinoma of the colon
QDot 655-gel formulation F4:
No membrane binding of QDot 655 in gel formulation F1 was detected in nonmalignant epithelia colon tissue after incubation for 1 hour at RT or 37° C.
A luminal membrane binding of QDot 655 in gel formulation F4 was detected in all three adenocarcinoma samples after incubation at RT (QDot 655 binding to 10%-32% of luminal membrane) or 37° C. (QDot 655 binding to 56%-83% of luminal membrane)
Intracellular binding (cytoplasm and nucleus) was observed in non-malignant epithelia colon tissue and adenocarcinoma of the colon
QDot 655-gel formulation F5:
No membrane binding of QDot 655 in gel formulation F5 was detected in nonmalignant colon tissue after incubation for 1 hour at RT or 37° C.
A luminal membrane binding of QDot 655 in gel formulation F5 was detected in all three adenocarcinoma samples after incubation at RT (QDot 655 binding to 7%-24%% of luminal membrane) or 37° C. (QDot 655 binding to 24%-28% of luminal membrane)
Intracellular binding (cytoplasm and nucleus) was observed in non-malignant colon tissue and adenocarcinoma of the colon

In summary, a membrane binding of QDot 655 using QDot 655-buffer, QDot 655-gel F4 or QDot 655-gel F5 in non-malignant epithelial tissue had never been observed. Membrane binding of QDot 665 using QDot 655-buffer, QDot 655-gel F4 or QDot 655-gel F5 had only been observed in cancer epithelium and was furthermore limited to the luminal membrane site. The observed luminal membrane binding pattern of QDot 655 using QDot 655-buffer, QDot 655-gel F4 or QDot 655-gel F5 is due to QDot 655 interaction with the luminal membrane site, since no such signal pattern had been observed in the respective controls PBS, Gel-F4 and Gel-F5. The difference of luminal QDot 655 binding using QDot 655-buffer, QDot 655-gel F4 or QDot 655-gel F5 between non-malignant and cancer epithelium is statistically significant (p<0.05).

No significant differences had been observed of QDot 655 luminal membrane reactivity in tumor between QDot 655-buffer, QDot 655-gel F4 or QDot 655-gel F5 at RT and 37° C. with the exception for QDot 655-gel F4 at 37° C. which exhibited a significantly higher luminal membrane reactivity compared to QDot 655-buffer and QDot 655 gel-F5. However, a statistical significant difference (p<0.05) at RT and 37° C. was observed between the luminal membrane reactivity of CDH17 and QDot 655 using QDot 655-buffer, QDot 655-gel F4 and QDot 655-gel F5, respectively. Furthermore data scattering was apparently lacking for CDH17 membrane and luminal membrane reactivity (below detection sensitivity). The observed reactivity of CDH17 was highly consistent from batch to batch and also between samples as expected for a standardized IHC assay. Using the same method, the opposite was observed for QDot 655 binding using QDot 655-buffer, QDot 655-gel F4 and QDot 655-gel F5, respectively. Here a significant data scattering between batches was observed indicating for the weak and unspecific nature of the QDot 655 interaction observed at the luminal membrane in the cancer epithelium for QDot 655-buffer, QDot 655-gel F4 and QDot 655-gel F5, respectively.

A statistically significant difference of QDot 655 luminal membrane reactivity between RT and 37° C. has not been observed for QDot 655-buffer and QDot 655-gel F5 however respectively observed for QDot 655-gel F4 (p<0.05). This observation might indicate for a formulation specific effect of F4 rather than for QDot 655 dependent effect and this finding is potentially relevant for the use as injectable endoscopy formulation into gut tissue at body temperature. This interpretation is supported by the finding that no such difference was observed for QDot 655 reactivity using a simple PBS diluted QDot 655 preparation (QDot 655-buffer).

The obvious difference in the binding pattern between non-malignant and cancer epithelium is most likely due to the difference in the composition of the cell membrane (i.e. expressed membrane proteins, lipid composition and glycosylation pattern etc.) observed between non-malignant and cancer epithelium. Furthermore the observed binding pattern to the luminal membrane in cancer epithelium is most likely of unspecific nature.

### Example 11: Measurement of Viscosity

Viscosity of gel-F4/F5 and the formulations QDot 655-gel F4/F5 has been determined at 24° C., 37° C. and 41° C.

For the determination of the viscosity for each measuring temperature a shear rate ramp was used, whereas the shear rate was continuously increased in a defined time window. With this test the flow behavior of the test items can be characterized.

### Results:

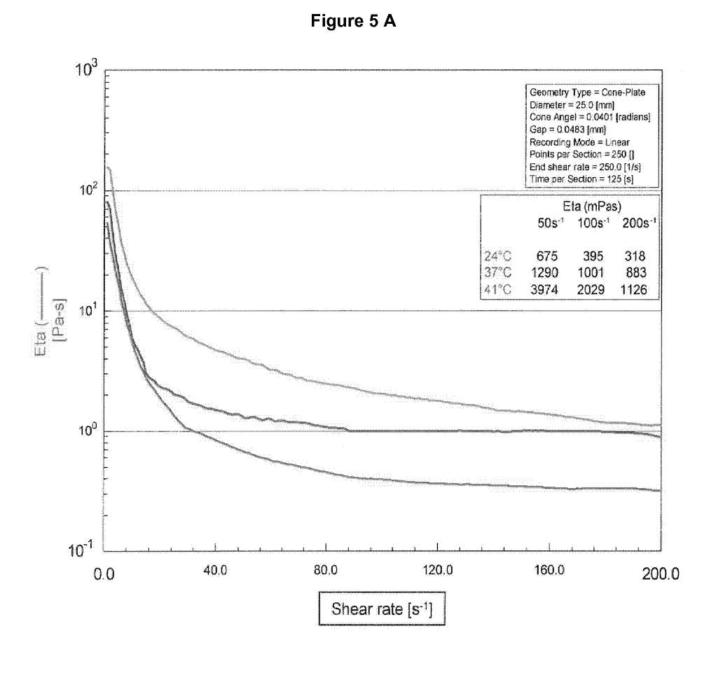

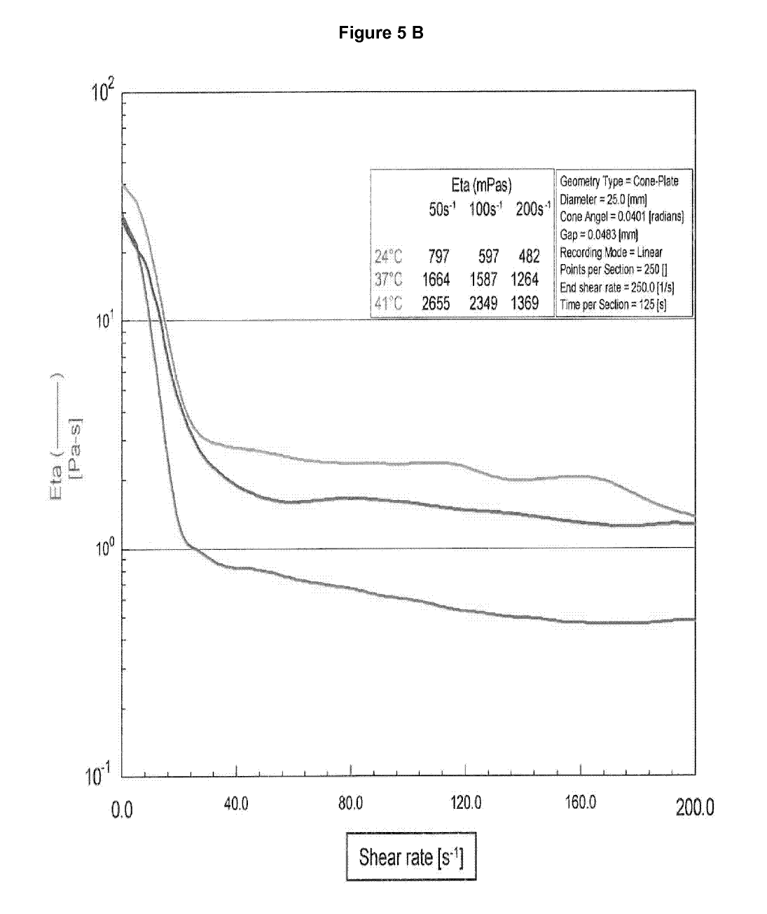

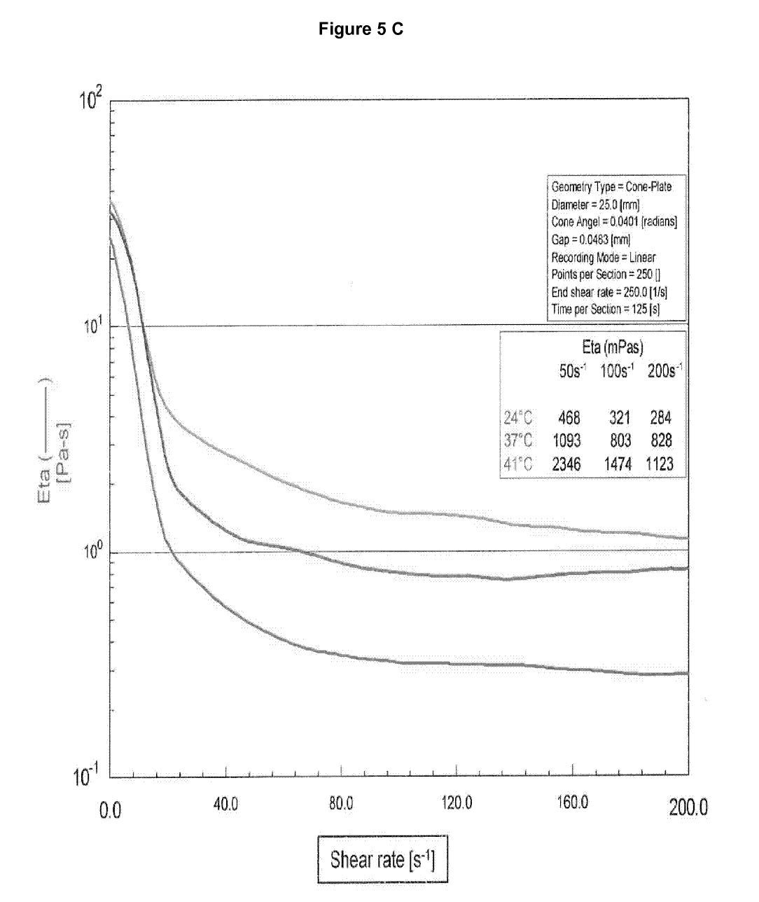

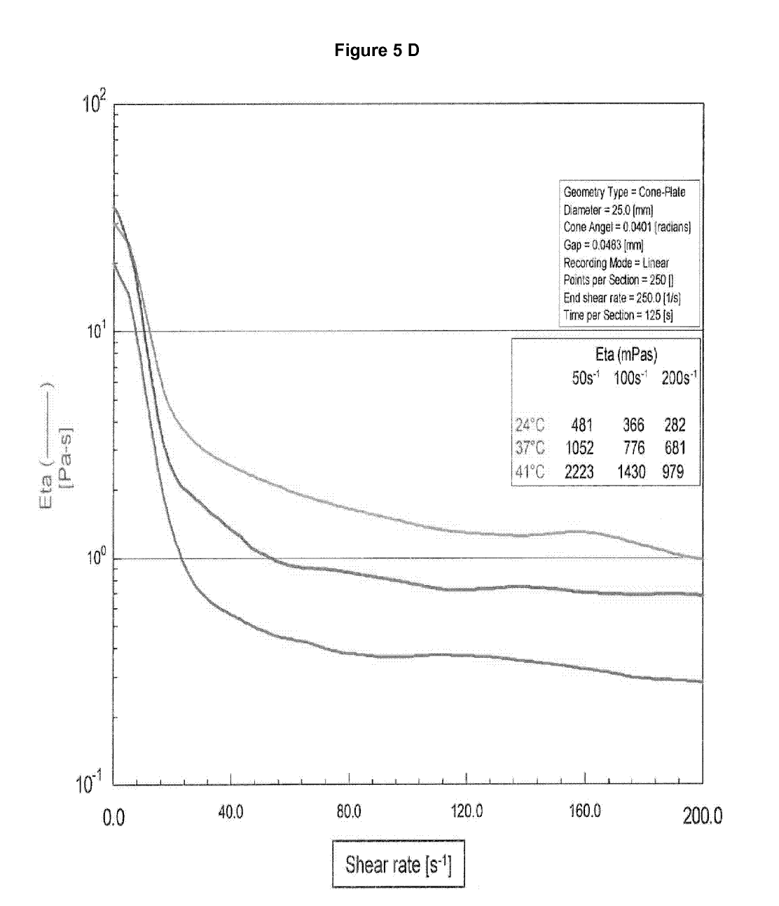

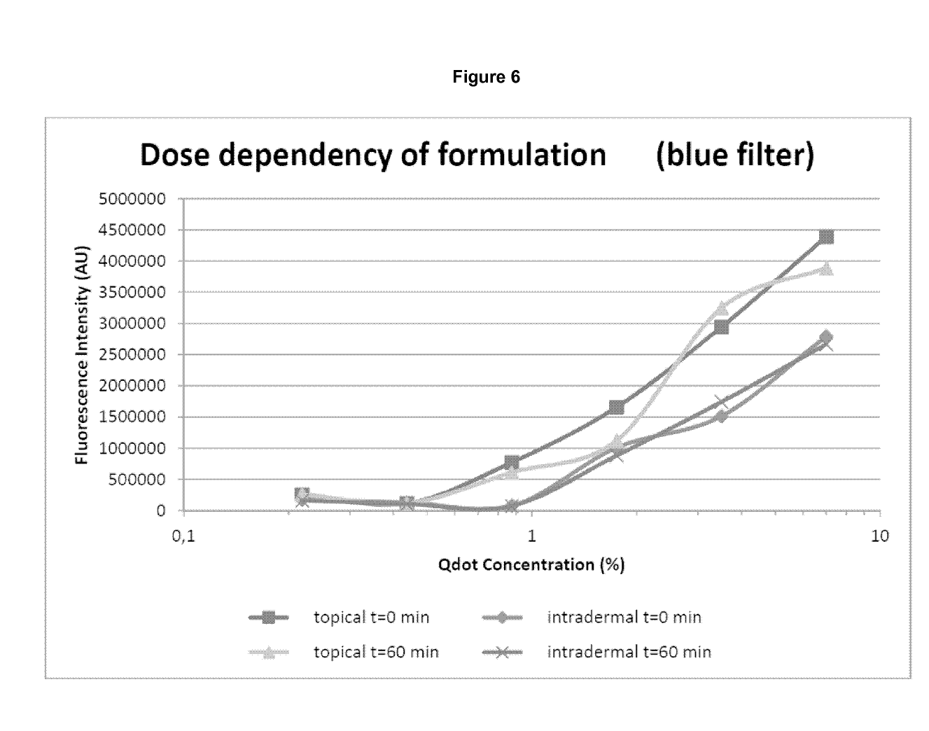

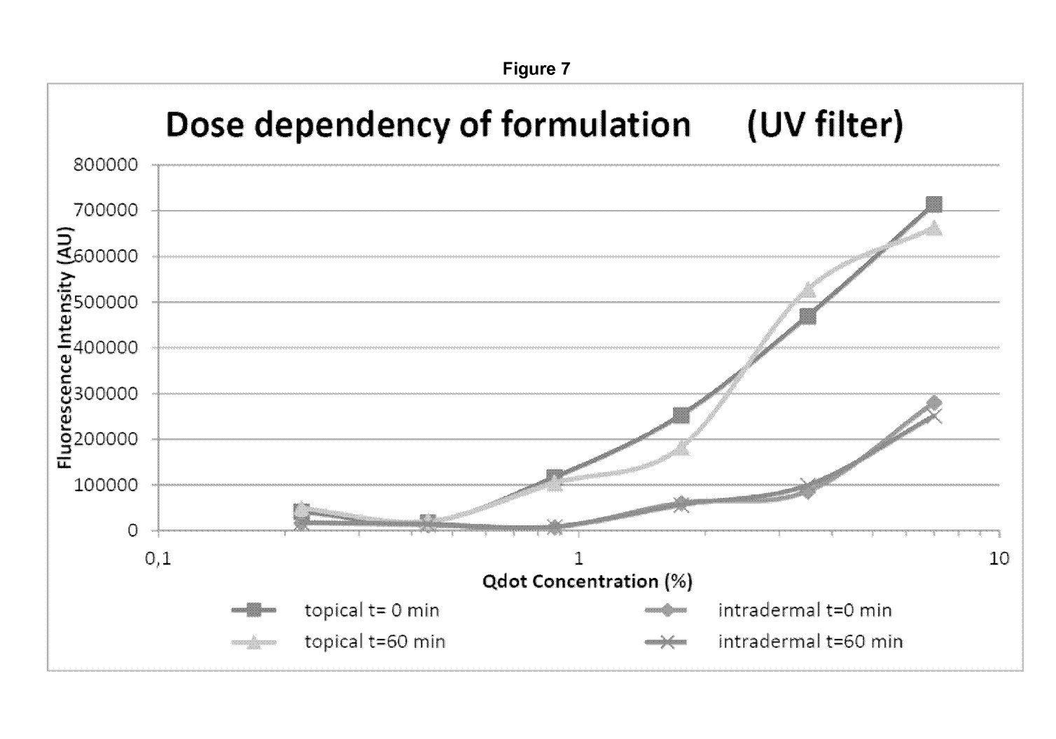

All test items show a structured flow behavior (see FIG. 5) that means with increasing shear rates the viscosity decreased. In the shear range of 1 s⁻¹ to ca. 50 s⁻¹ the decrease of the viscosities is extremely strong (from >10000 mPa to <4000 mPa). Afterwards the decrease of viscosities up to shear rate of 200 s⁻¹ is only moderate. The measurements show that there is an increase in the viscosities with increasing temperature from 37° C. to 41° C.

### Example 12: Detection of the Fluorescence Dose Dependency of the Inventive Formulation in Pig Gut Mucosa, In Vitro Evaluation of the QDot-Signal as a Location Marker for Colon Tissue Using the Maestro™ In-Vivo Fluorescence Imaging System

This study was performed based on the general principles of Good Scientific Practice (Good scientific practice in research and scholarship, European Foundation, December 2000), aimed at gaining knowledge based on verifiable results.

The objective of the project is to evaluate the dose dependency of the fluorescent Qdots in the inventive formulation applied by intra-mucosal injection (recommended praxis application; in the following “intradermal”) and onto the surface (application by default, in the following “topical”). This will be evaluated in a model using porcine colon material ex vivo. The minimally required dose of the material will be optically quantified, as well as the optimal dose of QDot ingredients.

### 1. Manufacturing Protocol of Tested QDot 655 Gel Formulation F6 and F7

### List of Substances and Equipment Used:

QDot®655 colloidal solution in 50 mM borate buffer from LTC Life Technologies Corporation, Carlsbad, Calif., US C47013

Kolliphor® P407 BTC Europe GmbH, Burgbernheim

Kolliphor® P188 BTC Europe GmbH, Burgbernheim

Aqua ad injectabilia Brau, Melsungen

PR2003 Delta Range Balance and AG 204 Balance: Mettler Toledo

Magnetic stirrer IKAMAG RCT: IKA, Staufen

Ultra Sonic bath: Bandelin Sonorex Super RK 255H, Berlin

Biofuge Stratos: Heraeus

Vivaspin Cartridge Turbo 15 MW 5000: Sartorius Stedim Biotech, Göttingen

TABLE 14
Composition of final 3.5% QDot containing formulation F6

| | | net weight |
|---|---|---|
| Excipients and substance | Concentration [%] | in 60 g |
| Kolliphor ® P407 | 18.00% w/w | 10.8 g |
| Kolliphor ® P188 | 5.00% w/w | 3.00 g |
| Aqua ad inj. | 73.50% w/w | 44.10 g |
| purified QDots ®655 | 3.50% w/w | 2.10 g |

The final concentration of quantum dots in the formulation F6 was 0.015% per weight.

TABLE 15
Composition of final 3.5% QDot containing formulation F7

| | | net weight |
|---|---|---|
| Excipients and substance | Concentration [%] | in 60 g |
| Kolliphor ® P407 | 18.00% w/w | 10.80 g |
| Kolliphor ® P188 | 10.00% w/w | 6.00 g |
| Aqua ad inj. | 68.50% w/w | 44.10 g |
| purified QDots ®655 | 3.50% w/w | 2.10 g |

The final concentration of quantum dots in the formulation F7 was 0.015% per weight.

### Preparation of Carrier Formulation

Composition for F6 formulation, net weight

| | |
|---|---|
| Kolliphor ® P407 | 90.0 g |
| Kolliphor ® P188 | 25.0 g |
| Aqua ad inj. | 350.0 g |

Composition for F7 formulation, net weight

| | |
|---|---|
| Kolliphor ® P407 | 90.0 g |
| Kolliphor ® P188 | 50.0 g |
| Aqua ad inj. | 325.0 g |

Firstly, the net weight of the glass beaker was saved as tara and Kolliphor P407 as well as Kolliphor P188 were weight into a glass beaker. This beaker was placed in a preheated (70° C.-80° C.) water bath until the Poloxamer mixture was completely melted. The appearance was: colorless, transparent, homogeneous, liquid.

Thereafter Aqua ad injectabilia was added to the molten mass and dispersed by stirring. Appearance: colorless, gel lumps

Immediately after dispersing, the preparation was stored under refrigerated conditions (2° C.-8° C.) until appearance is colorless, homogeneous and transparent.

Add Aqua ad injectabilia was added to obtain the final net weight of 465 g (taking the tare weight into consideration).

### Purification of QDot®655 Original Solution by Repeated Filtration Steps

To purify 14 ml of QDot®655 original solution 7 Vivaspin tubes are used.

The net weight of Vivaspin tubes (without screw cap) was saved as tare. 2 g of QDot®655 original solution were transferred each into one of 7 Vivaspin cartridges and 8 g of Aqua ad injectabilia was added into each. The closed cartridge was centrifuged with an angle rotor of bench top centrifuge at 3000 g for 30 min and at 21° C. The filtrate from the lower part of the cartridges was removed and collected. Subsequently, 8 g of aqua ad injectabilia was added to the QDot®655 concentrate in the upper compartment of the cartridge which was carefully resuspended completely with a pipette. The cartridge was centrifuged with the angle rotor of bench top centrifuge at 3000 g for 30 min and at 21° C. The filtrate from the lower part of the cartridges was removed and collected again. This washing step was repeated, so 8 g of aqua ad injectabilia was added to the QDot®655 concentrate which was carefully resuspended with a pipette. The cartridge was centrifuged with the angle rotor of bench top centrifuge at 3000 g for 30 min and at 21° C. The filtrate from the lower part of the cartridges was removed and collected again.

Aqua ad injectabilia was exactly added to the QDot®655 concentrate in the upper compartment of the cartridges (taking the tare weight into consideration) to obtain the initial weight of 2 g. After resuspension 14 g of purified QDots®655 were stored at 4° C. under light protection in a 50 ml glass vial.

### Addition of Purified QDot®655 Suspension to the Carrier Formulation

55.8 g of F7 or respectively F8 carrier formulation were weighted in a glass beaker. 2.1 g of purified QDot®655 suspension and 2.1 g of Aqua ad injectabilia were added. The beaker was placed on a magnetic stirrer which slowly mixed until the formulation appears homogeneous, orange red and transparent.

The product was transferred into brown glass vials, sealed and stored under protection from light at 2° C. to 8° C.

Analogously also F7 or respectively F8 formulation with differing amounts (given in % per weight) of purified QDot®655 suspension were prepared.

### 2. Material and Methods

### Test Items:

Porcine colon, freshly slaughtered,

Q-Dots formulations F7 and F8

### Reference Items:

PBS buffer, F7 or respectively F8 carrier formulation,

as positive control were the topical applications of each used. Quantitative

measurement data are directly comparable.

### Control Items

PBS buffer, F7 or respectively F8 carrier formulation

### Porcine Colon Preparation

The porcine colon was obtained from the animal facility of the RWTH Aachen University Clinic. The animal was freshly slaughtered and the complete organ was dissected. The colon was cleaned with water and subsequently washed in phosphate buffered saline (PBS). Subsequently, a piece of 18 cm colon was dissected and opened with a longitudinal cut. In a first experiment, the piece was directly prepared on a heating plate. Based on the experience made in this experiment, a specific support was constructed, on which the colon preparation could be fixed, so the application of samples could be better followed-up during the measurements. The heating plate was always adjusted to 37° C. During the assay, several dilutions of the formulations F7 and F8 were tested. Additional, the empty carrier formulation and PBS were used as negative controls.

The samples were either applied topical using a precision pipette, or injected intra-mucosal using a syringe, when the temperature of the colon was at 37° C. During the first assay, a specific 50 μl Hamilton syringe was used. In the second assay, a disposable plastic syringe with a volume of 1 ml was used. In both assays, 25 μl of sample were applied. In order to be able to apply 25 μl of the sample with the imprecise disposable syringe, 25 μl of sample were put into an Eppendorf tube using a precision pipette. The application of test material (at room temperature) was done either by injection into the 37° C. warm mucosa or by topical application (falling droplet). The dose escalation scheme was applied in parallel rows comparing the two different formulations F7 and F8, incl. empty carrier formulation (negative standard) and defined positive standards (QDot aqueous solutions).

The fluorescence intensity of the samples applied either topically or intra-mucosally was quantified using the Maestro™ In-Vivo Fluorescence Imaging System. The following excitation filters were used during the assay: (1) UV-A at 335-379 nm, (2) Blue at 445-490 nm. The sample stage was set to level 1, the illumination was set to level 1. Thus for both illumination and recording, settings allowed the maximal surface acquisition. The experiment was performed in two parts. In the first experiment, the exposure times were set to 100 ms, 200 ms, 500 ms, 2000 ms and 5000 ms. In the second assay, the exposure times were set to 10 ms, 50 ms, 200 ms, 1000 ms and 5000 ms.

The data are required and processed by the maestro imager version 2.2 Software supplied by Cambridge Research & Instrumentation, Inc. (CRi). The obtained data are further analyzed with the Excel. Illustrations were generated with the same software.

Data were measured in single measurements for each presented assay of part 1 and part 2. Fluorescence recording was obtained as complete spectral measurement between 515 nm and 800 nm in 10 nm steps. The MASTRO analyzer separates the specific fluorescence signal from the background signal.

### Results

The result is the fluorescence intensity, given in arbitrary units. Results show that the measurements using exposure times exceeding 500 ms are in saturation of the detector at highest concentrations, thus not resulting in linear results. Therefore, all results are shown for the 200 ms exposure time measurements. Results show, that within one hour, there is no decline of fluorescence intensity, as both curves for the fluorescence at t=0 min and t=60 min overlap completely. Unfortunately, longer incubations (planned were 24 h) were not possible, as the bacterial contamination of the piece of gut did not make it possible to measure the fluorescence with confidence.

In assay 1, Qdots were applied without the use of a specified grid support. The tracking of the Qdots was difficult and in the analysis, partial overlapping of the individual spots was detected. Thus, a special grid was designed, that made it possible to exactly track the position of the topical and intradermal applications, separately. Using this support, the gut was specifically fixed and each spot was applied into a specific field. Assay 2 was performed using this specified grid support. The subjective detectability of the particles was recorded additionally to the digital detection of the fluorescence intensity. The results are summarized in Table 18.

TABLE 16
Measurements of QDot ® fluorescence of a range of Qdot concentrations
using the blue filter at t = 0 min.

| | | Fluorescence | Fluorescence | |
|---|---|---|---|---|
| | | Intensity | Intensity | Fraction |
| Formulation | Dose | (Topical) | (Intradermal) | Intradermal/Topical |
| F7 | 7.00 | 4390220 | 2790930 | 0.64 |
| F7 | 3.50 | 2941200 | 1511870 | 0.51 |
| F7 | 1.75 | 1656200 | 1007740 | 0.61 |
| F7 | 0.88 | 772369 | 76650 | 0.10 |
| F7 | 0.44 | 114886 | 130820 | 1.14 |
| F7 | 0.22 | 255777 | 177411 | 0.69 |
| F7 | 0.00 | 23322 | 27267 | 1.17 |
| F7_PBS | 3.50 | 3597620 | 1145950 | 0.32 |
| F7_PBS | 0.00 | 23376 | 40240 | 1.72 |

TABLE 17
Measurements of QDot ® fluorescence of a range of Qdot
concentrations using the UV filter at t = 0 min.

| | | Fluorescence | Fluorescence | |
|---|---|---|---|---|
| | | Intensity | Intensity | Fraction |
| Formulation | Dose | (Topical) | (Intradermal) | Intradermal/Topical |
| F7 | 7.00 | 713721 | 280597 | 0.39 |
| F7 | 3.50 | 469171 | 86155.2 | 0.18 |
| F7 | 1.75 | 252678 | 60935.1 | 0.24 |
| F7 | 0.88 | 116936 | 77101.5 | 0.07 |
| F7 | 0.44 | 18678.9 | 12426.6 | 0.67 |
| F7 | 0.22 | 41442.2 | 17642.8 | 0.43 |
| F7 | 0.00 | 6055.7 | 6821.6 | 1.13 |
| F7_PBS | 3.50 | 507092 | 35178.3 | 0.07 |
| F7_PBS | 0.00 | 4921.3 | 6729.4 | 1.37 |

TABLE 18
Visibility rating of spots by eye, the emission was subjectively
evaluated through a specified long-pass filter filter (blue filter
set, measurement of fluorescence emission using long pass
filter 515 nm, in the range between 515 nm and 800 nm)

| | | | | | | | Filterset |
|---|---|---|---|---|---|---|---|
| | | | | | | | (exita- |
| Formulation | 7% | 3.5% | 1.75% | 0.88% | 0.44% | 0.22% | tion) |
| F7 (topical) | ++ | ++ | + | + | − | − | Blue |
| F7 | ++ | ++ | + | − | − | − | Blue |
| (intradermal) | | | | | | | |
| F7 (topical) | − | − | − | − | − | − | UV |
| F7 | − | − | − | − | − | − | UV |
| (intradermal) | | | | | | | |
| F8 (topical) | ++ | ++ | + | + | − | − | Blue |
| F8 | ++ | ++ | + | − | − | − | Blue |
| (intradermal) | | | | | | | |
| F8 (topical) | + | + | − | − | − | − | UV |
| F8 | − | − | − | − | − | − | UV |
| (intradermal) | | | | | | | |

TABLE 19
Measurements of QDot ® fluorescence of a range of Qdot concentrations
using the blue filter at t = 0 min.

| | | Fluorescence | Fluorescence | |
|---|---|---|---|---|
| | | Intensity | Intensity | Fraction |
| Formulation | Dose | (Topical) | (Intradermal) | Intradermal/Topical |
| F8 | 7.00 | 2549130 | 726565 | 0.29 |
| F8 | 3.50 | 1602470 | 602706 | 0.38 |
| F8 | 1.75 | 993550 | 381072 | 0.38 |
| F8 | 0.88 | 765175 | 105859 | 0.14 |
| F8 | 0.44 | 331333 | 44717 | 0.13 |
| F8 | 0.22 | 179249 | 65569 | 0.37 |
| F8 | 0.00 | 13967 | 11288 | 0.81 |
| F8_PBS | 3.50 | 839692 | 588380 | 0.70 |
| F8_PBS | 0.00 | 14689 | 20692 | 1.41 |

TABLE 20
Measurements of QDot ® fluorescence of a range of Qdot
concentrations using the UV filter at t = 0 min.

| | | Fluorescence | Fluorescence | |
|---|---|---|---|---|
| | | Intensity | Intensity | Fraction |
| Formulation | Dose | (Topical) | (Intradermal) | Intradermal/Topical |
| F8 | 7.00 | 445603 | 14516.5 | 0.03 |
| F8 | 3.50 | 309807 | 82201.8 | 0.27 |
| F8 | 1.75 | 193812 | 30142.4 | 0.16 |
| F8 | 0.88 | 133797 | 8300.2 | 0.06 |
| F8 | 0.44 | 59605.8 | 5741.6 | 0.10 |
| F8 | 0.22 | 32955.5 | 13874.8 | 0.42 |
| F8 | 0.00 | 4695.5 | 4313.2 | 0.92 |
| F8_PBS | 3.50 | 137577 | 19347.3 | 0.13 |
| F8_PBS | 0.00 | 5102.9 | 5997.4 | 1.18 |

The goal of this study is the determination of the optimal formulation and the optimal concentration of Qdots for intradermal application. In total, formulation F1 is superior to F8. This is based on the measurement of the samples in assay 1 and 2 (see also tables 16-20)

In assay 1, both fluorescence intensities at topical application are similar, but the intradermal application shows a significantly reduced fluorescence intensity of F2 in comparison to F1. This difference is in part also probably due to the more difficult injectability of F2 using a very fine syringe. This observation was similar in assay 2. In this case, the F2 injection had to be repeated at another locus in order to successfully inject the formulation. An important measure of the tissue penetration of the fluorescence of the particles is the fraction of fluorescence of the intradermal signal/topical application. Comparing the two excitation wave length, the blue excitation (445-490 nm) is clearly superior to the UV excitation (335-379 nm). Thus, it is clearly recommended to use excitation wave length of the blue filter set, corresponding to 445-490 nm. Secondly, the formulation F7 is superior to F8, as the fraction of intradermal/topical fraction of fluorescence intensity of F7 in assay 2 is around 60%, whereas the fraction of F8 is only approx. 30%.

Best visibility by eye was achieved at concentrations ranging from 0.015% to 0.075% per weight.

At intradermal application, the particles were clearly visible at a concentration of 0.075%. This concentration seems to be the most appropriate for the application in patient material, the concentration of 0.040% Qdots were only detectable at topical application.

### Example 13: Preparation, Analysis and Short Term Stability of Further Inventive Formulations

### Materials and Methods:

TABLE 21
Nanoparticles used; MFP = methylfluoresceinphosphate

| | | | | | |
|---|---|---|---|---|---|
| | | Catalog | | Absorp- | Emis- |
| Nanoparticle | Manufacturer | no. | Solvent | tion | sion |

| | | | | |
|---|---|---|---|---|
| [ZrO]^{2+[}MFP]²⁻ | Institut | Hepes | 476 | 517 |
| | f. Anorg. | Buffer | nm | nm |
| | Chemie, | 30 | | |
| | Karlsruher | mmol/L | | |
| | Institut für | | | |
| | Technologie, | | | |
| | Karlsruhe, | | | |
| | Germany | | | |

| | | | | |
|---|---|---|---|---|
| CANdots | Center for | M1101201 | water | Non |
| Series M | Applied | | | fluorescent |
| 12 nm | Nanotech- | | | |
| encapsulated | nology | | | |
| Iron oxide | GmbH, | | | |
| | Hamburg, | | | |
| | Germany | | | |

### Chemicals and Excipients:

The following materials were used. All materials were of analytical grade if not otherwise stated.

Kolliphor®P407—(Poloxamer 407); BASF, Germany 50259528; Lot: WPWJ554C

Kolliphor®P188—(Poloxamer 188); BASF, Germany 50259527; Lot: WPCI522B

Aqua ad injectabilia; Braun Melsungen PZN 8609338

1 Pas Standard viscosity oil; Malvern Instruments Ltd., UK Product no.: U2400

### Equipment:

PR2003 Delta Range; Mettler Toledo

AG 204 Analytical balance; Mettler Toledo

Kirsch Special 466 Refrigerator; Philipp Kirsch GmbH, Offenburg,

Viscometer Bohlin Visco 88; Malvern Instruments Ltd., UK

Cone and Plate equipment angle: 5.4° diameter: 30 mm

Bohlin software V6.32.1.2

Refrigerated/heating circulator K10 and DC10; Haake, Karlsruhe, Germany

Heraeus Vacutherm Heating Cabinet Thermo Fischer, Germany

Sonorex Super RK255H temperature controllable Ultrasonic bath; Bandelin, Berlin, Germany

Magnetic stirrer IKAMAG RCT; IKA, Staufen, Germany

UV lamp, NU-8 KL excicatation wavelength: 254 nm, 366 nm; Benda, Wiesloch, Germany

### Consumables

Glass injections vials 20 ml; Zscheille & Klinger 24020

Glass injections vials 10 ml; Zscheille & Klinger 24010

20 mm rubber stoppers; Zscheille & Klinger 1771/4104/40

Aluminium crimp seals with plastic discs; Zscheille & Klinger 75071

Glass centrifuge tubes, round bottom; Th. Geyer Assistent 950/1

Research Pipettes 5-200 μl and 200-1000 μl; Eppendorf 3111 000.157 and 3111 000.165

1-200 μl Pipette tips; Eppendorf

200-1000 μl Pipette tips; Eppendorf

### Methods

### 1. Preparation of F1 Carrier Gel (Batch 22-164)

Composition of F1 carrier gel net weight:

Kolliphor® P407 54.0 g

Kolliphor® P188 15.0 g

Aqua ad inj. 201.0 g

Kolliphor P407 and Kolliphor P188 was weight into a glass beaker. The beaker was placed in a preheated (70° C.-80° C.) water bath. After the Poloxamer mixture was completely melted the appearance was colorless, transparent, homogeneous, and liquid. After the solution has clarified, preheated (70° C.-80° C.) Aqua ad injectabilia was added to the molten mass and dispersed by stirring. Immediately after dispersing, the preparation was stored under refrigerated conditions (2° C.-8° C.) until the appearance was again colorless, homogeneous and transparent. Finally, Aqua ad injectabilia was added to obtain the final net weight of 270 g.

### 2. Detection of Viscosity

Dynamic viscosity was determined using a Bohlin Visco 88 viscometer equipped with cone and plate test geometry suitable for analysis of high viscosity fluids and pastes. Calibration was performed by measuring the calibration standard (1 Pas Standard Viscosity Oil) at 21° C. to ensure the calibration status of the instrument before measuring the F1 gel. Evaluation of the temperature dependent properties of the inventive formulations was performed applying continuous shear forces at a shear rate of 250 min⁻¹. Each total measurement consisted of 10 measured values detected within a measurement period of about 2.5 minutes. Mean values were documented. F1 gels containing nanoparticles in aqueous solvents were assessed at 21° C., 4° C. and 32° C. Formulations were applied onto the lower plate of the Bohlin Visco 88 viscometer at 21° C. After completion of the measurement at 21° C., temperature was set to 4° C., and followed by detection at 31° C. Once temperature of 32° C. was achieved the whole system was kept at these conditions for additional 30 minutes before the measurement was started. Viscosity of gels at a temperature of 37° C. was described by visual inspection and photo documentation.

### 3. Addition of Nanoparticles to F1 Carrier Gels

After filling carrier gel into 20 ml injection bottles, water was added and mixed into the gel by inserting an agitator and stirring the sample on a magnetic stirrer. After that, nanoparticle solutions were added in different amounts. Nanoparticles were used according to Table 22.

TABLE 22
Composition of batches prepared by addition of nanoparticle solutions to
carrier gel.

| | Carrier | | | | Carrier | | |
|---|---|---|---|---|---|---|---|
| Batch | gel | nanoparticle | | Aqua | gel | nanoparticle | Aqua |
| No. | [g] | [g] | Test item | [g] | % | % | % |
| 22- | 9 | 0.35 | CANdots Series M | 0.65 | 90 | | |
| 169 | | | 12 nm | | | | |
| 22- | 9 | 0.35 | [ZrO]²⁺[MFP]²⁻ | 0.65 | 90 | | |
| 170 | | | | | | | |
| 22- | 9 | 0.1 | CANdots Series M | 0.9 | 90 | | |
| 171 | | | 12 nm | | | | |
| 22- | 9 | 1 | [ZrO]²⁺[MFP]²⁻ | 0 | 90 | | |
| 172 | | | | | | | |

Pictures were taken after mixing the samples for 15 minutes at day light and exposure to UV light.

### 4. Short Term Stability of Nanoparticle Containing Gels

Stability of formulations was assessed by incubation of corresponding samples at 36° C. to 38° C. for 7 days (starting day 1 after preparation), followed by storage at 4° C. to 8° C. for 7 days. Samples were visually inspected immediately after preparation and on day 1 after production. Stable formulations were continuously monitored on day 2, 3, 6, 7 and 8 at 37° C. and after changing to 4° C. on day 10, 13, 14 and 15, documented by taking pictures. The following parameters were assessed:
viscosity: 1=liquid like water, 2=slightly viscous, 3=viscous to highly viscous, 4=semisolid
transparency: 1=transparent, 2=slightly turbid, 3=turbid
sedimentation/particle agglomeration: yes/no
color: description of color

### Results:

### Appearance:

CANdots series M 12 nm: dark brown solution, few agglomerates; non-fluorescent particles

[ZrO]²⁺[MFP]²⁻: bright yellow solution, color appears more concentrated at the bottom of the glass (slight sedimentation)

In a first approach [ZrO]²⁺[MFP]²⁻: nanoparticles were added in a concentration of 3.5%. The resulting F1 gel appeared homogeneous and very pale yellow at day light. At a radiation wavelength of 366 nm, (which is below the optimal absorption wavelength of [ZrO]²⁺[MFP]²⁻ particles) a pale yellow fluorescence was observed. Increase of nanoparticle concentration up to 10% was performed, too. This sample appeared homogenous and light yellow at day light. Irradiation with 366 nm revealed yellow fluorescence.

### Short Term Stability:

Short term stability at 37° C. followed by storage at 4° C. was performed with both samples types at the two concentrations each. On day 1 after preparation batches 22-169, 22-171, 22-170 and 22-172 (F1 gels containing two different nanoparticles in two concentrations each) were placed at 37° C. in an incubator for 7 days. On day 8 samples were transferred to the refrigerator for another 7 days.

Visual inspection is summarized in Table 5a and 5b.

The 4 batches appeared semi-solid at 37° C. Photos were taken immediately after withdrawal from the incubator on day 2,3,6,7 and 8 of storage and samples were put into the incubator immediately after the photography.

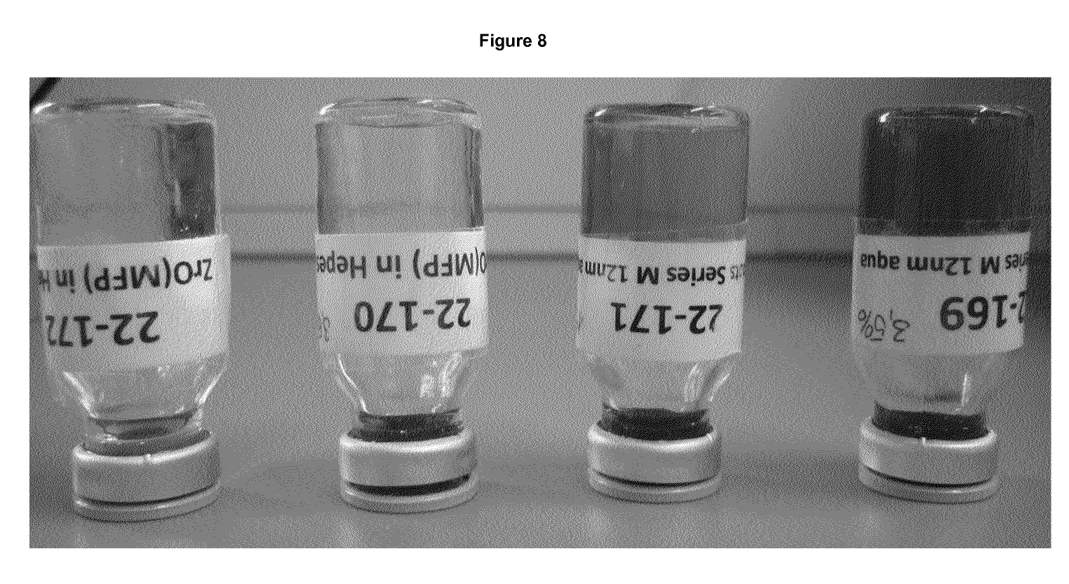

As a result of visual inspection, F1 gels containing 3.5% or 1% of CANdots Series M12, respectively, appear stable for a period of 7 days at 37° C. and F1 gels containing 3.5% or 10% of [ZrO]²⁺[MFP]²⁻ appear stable during a 7 day period of storage at 37° C. (cf. FIG. 8).

The tested F1 gels containing different amounts of nanoparticles in aqueous medium were liquid at 4° C. and room temperature. Batches 22-170 and 22-172, containing [ZrO]²⁺[MFP]²⁻ nanoparticles appear yellow, transparent and stable during 7 days of storage at 4° C. Exposure to UV light at a wavelength of 366 nm on day 8 and day 16 revealed fluorescence properties comparable to the day of preparation. Batches 22-169 and 22-171 containing CANdots Series M 12 nm appeared dark brown or brown, depending on the concentration of nanoparticles loaded into the gel. Appearance did not change after storage at 37° C. (FIG. 8) for 7 days. During storage at 4° C. slight sedimentation was shown in batch no. 22-169, containing 3.5% of iron oxide nanoparticles. However, sedimentation was not observed in sample 22-171, containing 1% of iron oxide nanoparticles within the 14 day stability test period.

### Discussion and Conclusion

Different nanoparticles in aqueous solution can be loaded to F1 gels as could be demonstrated with the iron oxide particles CANdots Series M12 and the [ZrO]²⁺[MFP]²⁻ in Hepes buffer.

Loading with 1% of non-fluorescent iron oxide particles resulted in a stable formulation. Dynamic viscosity did not significantly change as compared to empty F1 gel.

[ZrO]²⁺[MFP]²⁻ particles were tested in concentrations of 3.5% and 10%, respectively. Both formulations appeared stable with regard to qualitative fluorescence and the criteria described. Both formulations appear stable within the observation period described.

In contrast to the nanoparticles in aqueous media, it was not possible to load F1 carrier gels with nanoparticles in organic solutions (hexane and toluene). Stable formulations could not be achieved (phase separation took place). Furthermore, organic solvents affected strongly the viscosity characteristics of the resulting gels.

1. A pharmaceutical formulation for injection comprising 17% to 20% per weight of poloxamer 407 and 3%-15% per weight of poloxamer 188, 0.001%-0.15% per weight nanoparticles and water or an aqueous buffer.

2. The pharmaceutical formulation according to claim 1, comprising 3%-7% per weight of poloxamer 188.

3. The pharmaceutical formulation according to claim 1, wherein the nanoparticles are selected from the group consisting of: semiconductor nanoparticles, quantum dots, dye-doped silica particles, dye dope calcium particles, dye labeled liposomes, fluorescent carbon nanoparticles, iron oxide nanoparticles, gold nanoparticles, gold nanorods, gold nanoshells, nanoparticles containing organic chromophores, nanoparticles containing indocyanine green, silver/organic dye composites nanoparticles, nanoparticles containing a gadolinium oxide core, and combinations thereof.

4. The pharmaceutical formulation according to claim 3, wherein the nanoparticles are semiconductor nanoparticles or quantum dots.

5. The pharmaceutical formulation according to claim 4, wherein the quantum dots are cadmium selenide, zinc selenide or cadmium telluride quantum dots coated with zinc sulfide.

6. The pharmaceutical formulation according to claim 1, wherein the formulation is suitable for medical imaging.

7. The pharmaceutical formulation according claim 1, wherein the formulation comprises further at least one compound of the group consisting of monohydrogen phosphate, chloride, dihydrogen phosphate, tris(hydroxymethyl)aminomethane, citric acid monohydrate, trisodium citrate dehydrate, acetate trihydrate, glycine, bicarbonate, ammonia and borate.

8. The pharmaceutical formulation according to claim 1, wherein the nanoparticles contain a ligand selected from the group consisting of peptides, antibodies, and oligonucleotides.

9. The pharmaceutical formulation according to claim 1, comprising 18% per weight of poloxamer 407, 5% per weight of poloxamer 188, 0.01%-0.10% per weight quantum dots with an emission maximum of 655 nm and water.

10. A method for marking bodily areas of an organ or diseased body region comprising injecting the pharmaceutical formulation according to claim 1 directly into the organ or the diseased body region.

11. The method according to claim 10 for marking tumor areas.

12. The method according to claim 11, wherein the pharmaceutical formulation is used for marking gastrointestinal tumor areas by injection of the pharmaceutical formulation during an endoscopic or surgical procedure.

13. The method according to claim 10, wherein the pharmaceutical formulation is used for marking areas of skin to be discerned or removed during a surgical procedure.

14. The method according to claim 13 for marking areas in the dermis or mucosal layers of the skin to be discerned or removed during a surgical procedure.

15. A method for preparation of a pharmaceutical formulation according to claim 1 comprising the following steps:
a) dissolving 17% to 20% per weight of poloxamer 407 and 3%-15% per weight of poloxamer 188 in water;
b) stirring for at least 20 hours at 4° C.; and
c) adding at least one nanoparticle.
